(19) United States
(12) Patent Application Publication
(10) Pub. No.: US 2014/0187961 A1
(43) Pub. Date: Jul. 3, 2014

(54) CATHETER HAVING IMAGING FUNCTION, AND BLOOD VESSEL INSIDE OBSERVATION SYSTEM USING SAME

(71) Applicants: Kenichi Yamakoshi, Ishikawa (JP); Shinobu Tanaka, Ishikawa (JP)

(72) Inventors: Kenichi Yamakoshi, Ishikawa (JP); Shinobu Tanaka, Ishikawa (JP)

(21) Appl. No.: 14/008,690

(22) PCT Filed: Mar. 28, 2012
(86) PCT No.: PCT/JP2012/058197

§ 371 Date: Sep. 30, 2013

(30) Foreign Application Priority Data
Mar. 31, 2011 (JP) 2011-080732

(51) Int. Cl. A61B 1/00; A61B 8/12; A61B 1/12

(52) U.S. Cl. CPC A61B 1/00089; A61B 1/126; A61B 8/12
USPC 600/467; 600/146

(57) ABSTRACT

Hood 2 is formed to extend forward from the outer periphery of the tip of the tubular body 1. The tubular body is provided with at least a fluid delivery channel 11, an imaging channel 12 and a bending mechanism. Hood 2 has a basic shape of a cylindrical shape or a hollow circular truncated cone shape enlarging toward the tip, the tip of the hood has an obliquely-cut shape, and the hollow of the hood opens in the tip face 21 thereof. A hood tip 22 located at the tip of the wall part of the outer periphery of the tip of the hood bends to the inside of the hood and acts to stay the fluid.

### TECHNICAL FIELD

The present invention relates to a catheter having an imaging function, and an intravascular observation system for observation of the inside of a blood vessel.

### BACKGROUND ART

Among the various endoscopes (inclusive of catheters containing imaging function therein) to be inserted in living organisms, an endoscope to be inserted into the blood vessel sometimes has a device called balloon on the tip part thereof to interrupt the blood flow, so that an observation target site (intravascular wall and the like) can be seen (e.g., patent documents 1, 2 and the like).

Balloon is generally used along with a channel for intravascular injection of physiological saline.

As schematically shown in FIG. 18, a balloon 210 is swollen in the vicinity of the observation site in a blood vessel 100, as in the Figure, to interrupt blood 400, physiological saline 300 is intravascularly fed through a channel in an endoscope 200 to fill the blood vessel near the observation site with the saline, whereby visual observation through the imaging channel in the endoscope 200 is enabled.

On the other hand, cardiac disease patients are increasing in recent years due to the changes in lifestyle, aging and the like, along with which the demand for a treatment by intravascular insertion of a medical equipment such as catheter and the like (intravascular surgery) is also increasing. Intravascular surgery is advantageous in that it can be performed while visually observing the blood vessel lumen, and observing the detailed shape and color tone of the lesion with the naked eye, and is minimally invasive.

However, the present inventors have studied in detail the intravascular surgery including blood flow interruption with balloon, and found the following problem.

The problem is that, in an intravascular surgery including interruption of blood flow with balloon and substitution of blood with physiological saline to enable visual observation of the intravascular wall, long-time observation is not available, since it requires prevention of blood flow to the observation site by inflating the balloon located on the upstream side of the blood flow. In large blood vessels such as aorta and the like, moreover, application of balloon interrupts the blood flow from the heart (left ventricle) and blocks the systemic blood circulation. Therefore, application of balloon is difficult; hence, a treatment with visual observation is also difficult.

To solve the above-mentioned problem, the present inventors first provided a cylindrical-shaped hood (hood, cover) 600 on the tip of an endoscope body 500, as shown in FIG. 19(a), wherein the shape of the tip opening of the hood was that of an obliquely-cut cylindrical-shaped body of the hood. Furthermore, as shown in FIG. 19(b), they established a constitution wherein the tip of the endoscope body 500 inside a blood vessel 100 is bent so that the hood 600 will enclose an observation target site, a transparent fluid (e.g., physiological saline) 310 is injected into the hood from a fluid injection channel 510, and the observation target site on a blood vessel inner wall is imaged by an imaging channel 520 (hereinafter this endoscope is also referred to as a “hooded endoscope”).

Using the hooded endoscope, as shown in FIG. 19(b), the transparent fluid (physiological saline) 310 is injected into the hood from the fluid injection channel 510, and an opaque fluid (blood and the like) 410 surrounding the observation target site can be effectively removed even with a smaller injection amount as compared to balloon, and the observation target site can be observed visually.

However, the present inventors have verified in more detail the observation capability of the above-mentioned hooded endoscope they proposed and noted the following points yet to be improved.

(a) It is ideal in the above-mentioned hooded endoscope to tightly enclose the observation target site with the hood so that an opaque fluid such as blood and the like will not enter into the hood from the periphery. In actual operation of an endoscope, however, it is difficult to bring the whole circumference of the hood opening in close contact with the wall surface around the observation target site, as in FIG. 19(b). As a result, the hood opening is released slightly from the wall surface of the observation target site and an opaque fluid such as blood and the like easily enters into the hood from the gap to prevent the view. Therefore, a relatively large amount of physiological saline needs to be discharged to prevent the blood from entering into the hood. In addition, there is a clinical demand for continuous observation of the blood vessel inner wall while rotating the endoscope in the circumferential direction (direction around the outer circumference of the body about a tubular axis). In this case, the observation needs to be continued by intentionally setting apart the hood opening slightly from the wall surface of the observation target site while rotating the endoscope. Again, a relatively large amount of physiological saline needs to be discharged to prevent the blood from entering into the hood.

(b) When a transparent fluid is injected into the hood, the transparent fluid flows out from the hood through the gap between the hood and the observation target site. In this case, as shown in FIG. 20 with a thick arrow, the injected transparent fluid smoothly flows out from the tip of the hood to the outside due to the force of injection, which prevents the inside of the whole hood from being efficiently transparent. This phenomenon is not markedly improved by changing the position of the fluid injection channel. To make the whole inside of the hood transparent, injection of a relatively large amount of a transparent fluid is necessary, though not as much as the balloon of FIG. 18.

### DOCUMENT LIST

### Patent Documents

patent document 1: JP-A-2002-112954

patent document 2: JP-A-2007-289231

patent document 3: JP-A-H10-514603

patent document 4: WO1999/049910

patent document 5: WO2006/000942

### SUMMARY OF THE INVENTION

### Problems to be Solved by the Invention

The purpose of the present invention is to solve the above-mentioned problem, and provide an equipment that enables visualization of an observation target site with a less amount of a transparent fluid even in a suspended fluid such as inside of a blood vessel, and a system for effectively utilizing the equipment.

### Means of Solving the Problems

The present inventors have conducted intensive studies in an attempt to further improve the above-mentioned hooded endoscope they proposed and found that, in a structure wherein at least a part on the tip side in the whole circumference of the opening of the hood is bent toward the inside of the hood, a transparent fluid injected in the hood hits the bent part and stays in the hood and near the opening, whereby the inside of the hood can be effectively made transparent even when the opening of the hood is apart from the observation target site and the injection amount is small, which resulted in the completion of the present invention.

Accordingly, the main constitution of the present invention is as follows.
(1) A catheter having an endoscope function, comprising:

a tubular body, and

a hood extending forward from the outer periphery of the tip of the tubular body, wherein

the aforementioned tubular body comprises at least a fluid delivery channel for injecting forward a fluid from the tip of the tubular body, and an imaging channel for observation of the external environment from the tip of the tubular body, and a bending mechanism capable of bending a section with a predetermined length from the tip of the tubular body toward at least one lateral direction is provided,

the aforementioned hood has a basic shape of a cylindrical shape, or a hollow circular truncated cone shape enlarging toward the tip, the tip of the hood has a shape obtained by obliquely-cutting the aforementioned basic shape, the hollow in the hood opens at the tip face thereof and, in addition thereto, in the wall part of the outer periphery of the tip of the hood, at least a part located at the tip is bent toward the inside of the hood to prevent the flow of the aforementioned fluid heading toward the outside from a part located at the tip of the aforementioned opening.
(2) The catheter of the above-mentioned (1), wherein the basic shape of the hood tip is a shape obtained by cutting the cylindrical shape or circular truncated cone shape, which is the basic shape of the hood, with a flat plane forming an angle of 30 degrees −60 degrees to the central axis thereof.
(3) The catheter of the above-mentioned (1) or (2), wherein the basic shape of the hood is a cylindrical shape having the same outer diameter as that of the tubular body.
(4) The catheter of the above-mentioned (1) or (2), wherein the basic shape of the hood is a hollow circular truncated cone shape enlarging toward the tip,

the outer diameter of the tip of the tubular body is narrower than the outer diameter of the body from the proximal part to an intermediate part of the tubular body, and the hood extends enlarging forward from the outer periphery of the tip,

the maximum outer diameter of the tip of the hood is the same as the outer diameter of the body from the proximal part to the intermediate part of the tubular body.
(5) The catheter of the above-mentioned (4), wherein an endoscope with an imaging channel is inserted in the inside of the tubular body of the catheter, a narrow part of the tip of the tubular body secures at least a fluid delivery channel and holds the body of the aforementioned endoscope.
(6) The catheter of any of the above-mentioned (1)-(5), wherein

the bending direction by the bending mechanism and the direction of inclination of the hood opening are related such that the tip of the tubular body part bends toward the direction that brings a line segment connecting A and B closer to parallel to the central axis of the catheter or a direction opposite therefrom, wherein A is a point located at the tip side of the outer periphery of the opening at the tip of the hood and B is a point located at a rear end side thereof.
(7) The catheter of any of the above-mentioned (1)-(6), further comprising one or more ultrasonic oscillators, which act as ultrasonic wave transmitting-receiving elements, on the main body of the tubular body of the catheter, to enable an ultrasonic diagnosis of the observation object inner wall for which the catheter is inserted.
(8) The catheter of the above-mentioned (7), wherein a plurality of ultrasonic oscillators are provided as an electronic phased array.
(9) An intravascular observation system comprising at least the catheter of any of the above-mentioned (1)-(8),

a fluid delivery device for delivering a transparent fluid to the fluid delivery channel to inject the transparent fluid from the tip of the fluid delivery channel contained in the tubular body of the catheter, and

a control device for controlling driving of the fluid delivery device, wherein

the fluid delivery device is constituted to be controlled by the control device to perform or stop delivery of the fluid to the fluid delivery channel, and

the control device is constituted to receive a signal indicating the heart motion of a patient to be inserted with the aforementioned catheter as an input signal, and control the fluid delivery device based on the input signal to inject a predetermined amount of the transparent fluid from the tip of the fluid delivery channel of the aforementioned catheter, during the period when the blood flow stops at the tip of the catheter.
(10) The intravascular observation system of the above-mentioned (9), wherein the imaging channel contained in the tubular body of the aforementioned catheter is constituted to start imaging of the observation target site in synchronization with the timing of injection of the transparent fluid from the fluid delivery channel and perform imaging only for a predetermined time, or

while an imaging channel in a tubular body contained in the catheter is constantly imaging the observation target site, it is constituted such that the control device starts recording of images in synchronization with the timing of the injection of a transparent fluid from the fluid delivery channel and performs recording of images only for a predetermined time.
(11) The intravascular observation system of the above-mentioned (9), wherein the imaging channel contained in the tubular body of the aforementioned catheter is constituted to start imaging of the observation target site in synchronization with the timing of injection of the transparent fluid from the fluid delivery channel and perform imaging only for a predetermined time, or

while an imaging channel in a tubular body contained in the catheter is constantly imaging the observation target site, it is constituted such that the control device starts recording of images in synchronization with the timing of the injection of a transparent fluid from the fluid delivery channel and performs recording of images only for a predetermined time.
(12) The intravascular observation system of the above-mentioned (9), further comprising one or more ultrasonic oscillators, which act as ultrasonic wave transmitting-receiving elements, on the main body of the tubular body of the catheter, to enable an ultrasonic diagnosis of the observation object inner wall for which the catheter is inserted.
(13) The intravascular observation system of the above-mentioned (12), wherein a plurality of ultrasonic oscillators are provided as an electronic phased array.

### Effect of the Invention

The “catheter” in the present invention is a [hollow, tubular equipment to be inserted into cavity, lumen, blood vessel and the like]. It may be a tubular-shaped medical equipment to be inserted in the body, as well as a tubular equipment insertable into an article.

The “channel” in the present invention means a pathway constituted to convey the object action such as conduit line, electrical line, waveguide line, heat transfer line and the like, and includes not only a simple transmission pathway but also a device and a structure constituted to perform the action such as [electrical line connecting a proximal end and a tip, and a tip light emitting (imaging) device].

The “imaging channel” in the present invention means a pathway and a device such as a camera at the tip and electric line or an optical fiber and the like, which are constituted to take images at the tip and send the obtained images to a proximal part.

The “fluid injection channel” in the present invention means a pathway such as conduit line, space and the like and a device, which are constituted to deliver a fluid sent from a proximal part to the tip side and allow discharge from the tip.

The “catheter having an endoscope function” in the present invention may be a catheter as a tubular equipment provided with imaging parts therein to exhibit an endoscope function, or a catheter inserted with an endoscope as an independent product therein, or an endoscope itself. Therefore, a constitution wherein an imaging channel and a fluid delivery channel are provided in a tubular body more specifically includes, but is not limited to, the following embodiment, and any combination can be adopted.

(A) An embodiment wherein the tubular body is a simple tube, the imaging channel is an independent endoscope, and the fluid delivery channel is an independent tube for fluid delivery.

(B) An embodiment wherein the tubular body is a simple tube, the imaging channel is an independent endoscope, and the fluid delivery channel is the remaining space in the tubular body.

(C) An embodiment wherein the tubular body is an outside tube of an endoscope, and a fluid delivery channel is formed in the endoscope. In this case, the catheter can also be said to be an endoscope having a fluid delivery channel therein.

Among the above-mentioned various embodiments, the embodiments of the above-mentioned (A), (B) wherein an endoscope having an ultrafine diameter is inserted into a simple tube used as a catheter are preferable, since more existing products can be utilized as parts, production is easy because the structure is simpler as compared to the one shown in FIG. 19, and the cost can be reduced in terms of the aspects of parts and materials, assembly, and maintenance.

The catheter according to the present invention is provided with a hood having a cylindrical shape or circular truncated cone shape as a basic shape at the tip of the tubular body, and the tip has an obliquely-cut shape. As a result, as shown in FIG. 1, a tip part of tubular body 1 is bent to bring the opening of hood 2 closer to or in contact with an observation target site, a transparent fluid (e.g., physiological saline) is discharged from a fluid injection channel 11, and the periphery of the observation target site can be effectively made transparent even with a small discharge amount, due to the hood covering, whereby the observation target site can be observed visually.

Furthermore, in the catheter according to the present invention, as shown in FIG. 1, in the wall part of the outer periphery of the tip of a hood 2, at least a part 22 located at the tip (hereinafter this part is also referred to as a “hood tip”) is bent toward the inside of the hood 2 to prevent the flow of the aforementioned fluid f heading toward the outside from a part located at the tip of the opening.

By bending the hood tip as mentioned above, the transparent fluid f injected from a fluid injection channel 11 is deprived of the smooth flow out as in FIG. 20, made to turn at the bending part in the hood tip and head toward the observation target site, and stays turbulent near the opening of the hood, as shown in FIG. 1 with a thick arrow. Since the transparent fluid f stays near the opening, as in FIG. 1, the periphery of the observation target site can be effectively made transparent even when an opening surface 21 of the hood is about 1 mm apart from the wall surface of the observation target site and even with a small injection amount, and the observation target site can be observed visually.

Moreover, as shown in FIG. 3(a), when the basic shape of the hood is a circular truncated cone shape with an increasing inner diameter toward the tip, and the flow stays at the opening, the field of view spreads during imaging and the observation target site can be observed visually.

When the catheter according to the present invention is applied as an endoscope for intravascular observation, the present invention takes note of the point at which the blood flow changes due to the motion of the heart (particularly, the moment when the blood flow almost stops due to cardiac diastole), and proposes to effectively make the periphery of an observation target site transparent by an injection of a small amount of a transparent fluid, by injecting a predetermined amount of a transparent fluid at a moment when the blood flow almost stops.

In the intravascular observation system according to the present invention, the control system is constituted such that the aforementioned [injection of a transparent fluid synchronized with the moment when the blood flow almost stops] can be achieved, wherein a motion signal of the heart is an input, and an appropriate amount of a transparent fluid is injected in the hood at an appropriate timing of the moment when the blood flow almost stops to effectively make the periphery of an observation target site transparent, thus enabling imaging.

In a preferable control constitution of the present invention, imaging or recording of images can be performed in synchronization with the aforementioned injection of a transparent fluid, namely, at the timing when the hood is made transparent.

### BRIEF DESCRIPTION OF THE DRAWINGS

FIG. 1 shows the constitution of the catheter of the present invention, and is a cross-sectional view of the tip portion along the central axis of the catheter. Detailed structures of respective channels and mechanisms such as an imaging channel in a tubular body, a bending mechanism and the like are omitted.

FIG. 2 is a schematic view of a cross-section of the tip portion of the catheter of the present invention, and particularly explains the manner of bending of the hood tip and the shape and size of respective parts. This Figure is a cross-sectional view obtained by cutting with a flat plane containing the central axis Y of the tubular body, point A at the tip of the outer periphery of the opening of the hood, and point B at the rear end thereof. This manner of cutting is the same as in FIG. 1, FIG. 3, FIG. 19(b), FIG. 20.

FIG. 3 is a cross-sectional view showing an embodiment of the hood enlarging in a circular truncated cone shape. FIGS. 3(b), (c) each show a cross-sectional view along X-X of FIG. 3(a).

FIG. 4 is a cross-sectional view showing another constitution of the catheter according to the present invention. In the embodiment of this Figure, an ultrasonic oscillator for intravascular ultrasonic diagnostics is provided in the tubular body of the catheter. In FIG. 4(a), only the ultrasonic oscillator shows its outside rather than a cross-section thereof. In FIG. 4(b), the ultrasonic oscillator also shows its cross-section but the detailed inside structure is omitted.

FIG. 5 shows one embodiment of the constitution of the intravascular observation system of the present invention. FIG. 5(a) is a block diagram showing the connection relationship of each device constituting the system, and FIG. 5(b) is a time chart showing an input signal and action of each part, giving an example of the operation method of the system.

FIG. 6 is a schematic view explaining the constitution of the experiment facility for confirming the action effect of the catheter and the intravascular observation system of the present invention.

FIG. 7 shows the size and specification of a target pattern configured as an observation object in a piping in the experiment facility of FIG. 6.

FIG. 8 is a graph chart showing the experiment results confirming the action effect of the catheter and the intravascular observation system of the present invention.

FIG. 9 is a graph chart showing the experiment results confirming the action effect of the catheter and the intravascular observation system of the present invention.

FIG. 10 shows an Example for confirming the action effect of the catheter and the intravascular observation system of the present invention.

FIG. 11 shows the results of an Example for confirming the action effect of the catheter and the intravascular observation system of the present invention.

FIG. 12 shows the results of other Example for confirming the action effect of the catheter and the intravascular observation system of the present invention.

FIG. 13 shows other Example for confirming the action effect of the catheter and the intravascular observation system of the present invention.

FIG. 14 shows the results of other Example for confirming the action effect of the catheter and the intravascular observation system of the present invention.

FIG. 15 shows the constitution of the catheter produced in Example 5.

FIG. 16 is a graph chart showing the results of the static characteristic test and dynamic characteristics test performed in Example 5.

FIG. 17 is a graph chart showing the results of the experiment confirming the action effect of the catheter and the intravascular observation system produced in Example 5.

FIG. 18 schematically shows the use state of a conventional endoscope with balloon.

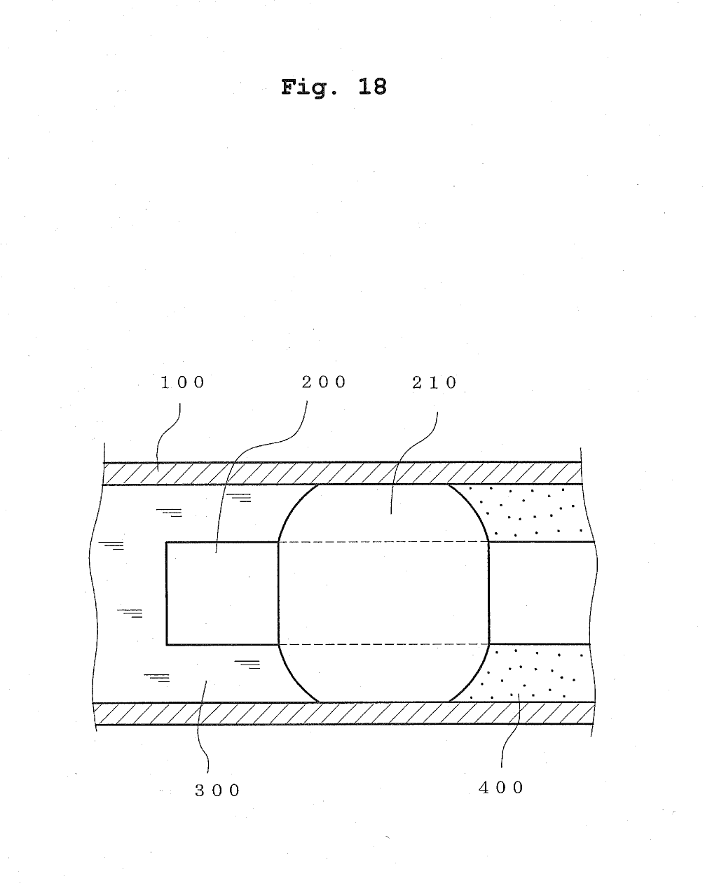

FIG. 19 shows the constitution of a hooded endoscope proposed by the present inventors in an attempt to solve the problem of an endoscope with balloon.

FIG. 20 shows the problems to be improved in the hooded endoscope shown in FIG. 19, which were found by the present inventors.

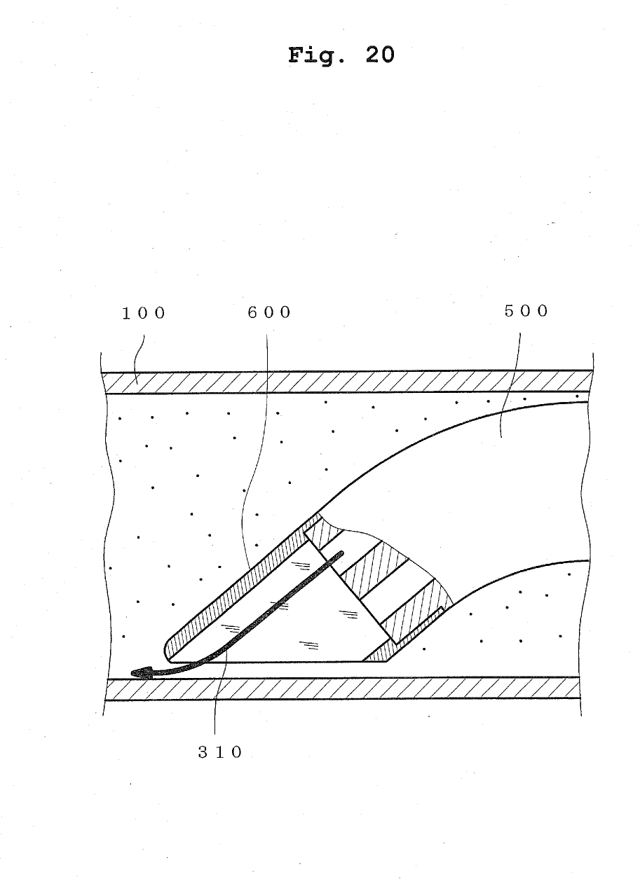

### DESCRIPTION OF EMBODIMENTS

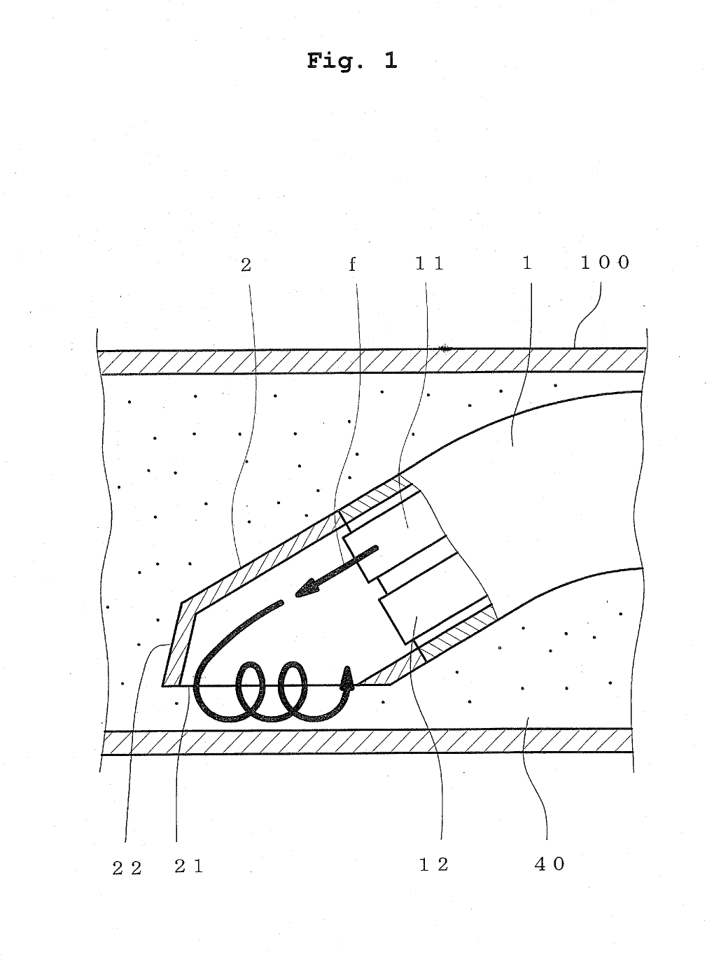

The catheter according to the present invention is constituted to have, as shown in FIG. 1, a tubular body 1, and a hood 2 extending forward from a tip thereof.

As shown in FIG. 1, the tubular body 1 is provided with at least a fluid delivery channel 11 and an imaging channel 12 in the inside thereof. The fluid delivery channel 11 is constituted to send a transparent fluid f from a proximal operating part (not shown) and inject same from a tip face, and the imaging channel 12 is constituted to send the images of the observation target site. The “imaging channel” in the present invention means a device that captures images ahead the tip side and sends them to the proximal side. With this imaging channel, the catheter has an endoscope function. As shown in FIG. 1, moreover, the tubular body 1 has a bending mechanism capable of bending the tip thereof to at least one lateral direction (downward in FIG. 1).

The basic shape of the hood 2 is a cylindrical shape, or a hollow circular truncated cone shape enlarging toward the tip, as in FIG. 1. The hood 2 extends forward from a tip of the tubular body 1.

The fluid injection channel 11 delivers the transparent fluid f into the hood, and the imaging channel 12 takes photographs of the external environment from within the hood. The tip of the hood 2 has a shape obtained by obliquely-cutting the aforementioned basic cylindrical shape or circular truncated cone shape (cylindrical shape in the example of Figure), and the hollow in the hood opens at the tip face thereof. In the present invention, the hood is further deformed, and the hood tip 22 is bent toward the inside of the hood to prevent the flow of the aforementioned transparent fluid f heading toward the outside from a part located at the tip of the aforementioned opening.

By the above-mentioned constitution, as explained in the above-mentioned Effect of the Invention, the tip of the tubular body 1 can be bent to position the hood to cover an observation target site and, in this state, a transparent fluid (physiological saline and the like) f is injected in the hood from the fluid injection channel 11. As a result, the flow thereof hits a hood tip 22 and stays, for example, as shown by the thick arrow in FIG. 1. Since the fluid stays, an opaque fluid (blood and the like) 40 in the periphery of the observation target site can be effectively removed even with a small injection amount, and the observation target site can be photographed by the imaging channel 12.

The catheter can be used not only for medicine but also for any application including visual observation, from the outside, of an observation target site in an opaque fluid. The catheter is preferable for the observation of various parts in the body, inter alia, use in blood vessels, and particularly useful for large blood vessels where use of balloon is not preferable, such as ascending aorta, aortic arch, descending aorta and the like.

To know the basic internal structure of each part from the tip of the imaging channel and fluid delivery channel to the proximal operation parts thereof, the mechanism for transmitting various information of the tip to the proximal side, the mechanism for transmitting operation on the proximal side to the tip side and the like of the catheter, the mechanisms mounted on conventionally-known catheters and endoscopes can be referred to (e.g., patent document 3 and the like).

At least a fluid injection channel and an imaging channel are formed in the tubular body. It is preferable to add a lighting channel for imaging.

When not only images of an observation target site is simply taken but also various in situ treatments are performed on the observation target site (mechanical treatment for cutting, partial removal and the like of an observation target site, treatments such as heating, application of electric current, voltage, irradiation of laser beam, medication, fluorescence observation, obtaining tomographic image and the like with respect to an observation target site), various optical fibers and channels may be added such as forceps channel, special light irradiation fiber, Optical Coherence Tomography (OCT) probe channel and the like.

The outer diameter and inner diameter of the main body of the tubular body are not particularly limited. For industrial use such as observation of the inside of a piping and the like, the maximum outer diameter is about 15 mm, maximum inner diameter is about 10 mm; for medical use, the maximum outer diameter is about 10 mm, and the maximum inner diameter is about 8 mm. Among others, for intravascular observation use, the maximum outer diameter of the body of the tubular body is about 6 mm, and the maximum inner diameter is about 5 mm, more preferably, the maximum outer diameter is about 5 mm and more preferable maximum inner diameter is about 4 mm.

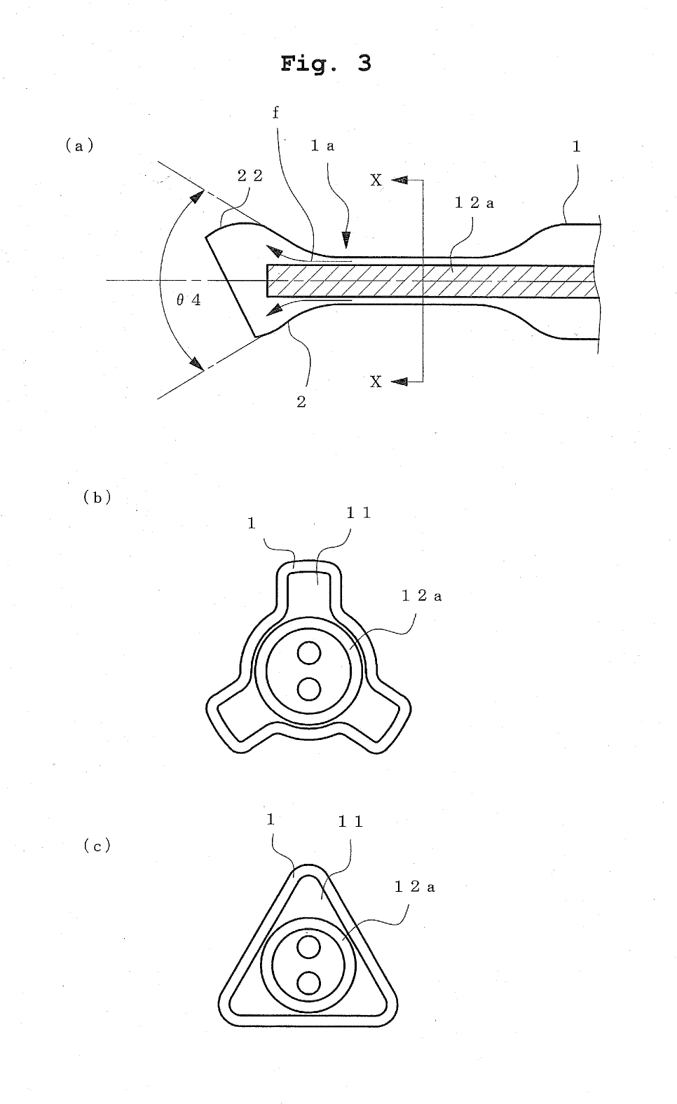

On the other hand, the minimum diameter of the main body of the tubular body only needs to be a size permitting provision of an endoscope function in the inside, or permitting insertion of an endoscope. When a bending mechanism, an imaging channel, and a fluid delivery channel are set inside thereof, the minimum outer diameter of the tubular body at this point is about 6 mm, and the minimum inner diameter is about 5 mm. With the progress of the technique for making the diameter thinner, the minimum diameter of the tubular body may be reduced as appropriate. When a topically narrow or thick part needs to be formed to meet the design needs, as in the embodiment shown in FIG. 3 and the like, the outer diameter may be appropriately changed regardless of the aforementioned preferable ranges.

The inner diameter may be changed as appropriate according to the strength of the material of the tubular body.

For the purpose of intravascular observation, a preferable minimum outer diameter of the main body of the tubular body is about 3 mm, practically more preferably about 4 mm, in view of the mechanical strength of the hood, the structure of the connection part between the tubular body and the hood, fluid resistance of the fluid delivery channel and the like.

While the material of the tubular body is not particularly limited, when it is used as a general medical catheter, polyurethane, polyester, polyolefin, fluorine-based resin (e.g., Teflon (registered trade mark)), nylon, silicone rubber, and the like are preferable materials.

While the tubular body may be formed from the same material over the full-length, the material may be changed depending on the part. For example, a flexible material may be used for a part to be bent by the bending mechanism, a highly rigid material is used for other parts and the like.

When the fluid delivery channel is an independent tube, the inner diameter thereof can be appropriately determined depending on the outer diameter of the main body of the tubular body, and the presence of other channels.

For example, for the purpose of intravascular observation, a preferable outer diameter of the main body of the tubular body is 4 mm-6 mm, as mentioned above, and a preferable inner diameter of the fluid delivery channel in this case is about 1 mm-2 mm.

The injection port at the tip of the fluid delivery channel may be a simple opening, or a nozzle or orifice having an intended increase in the angle and flow rate, which is designed to be able to discharge a transparent fluid.

When the fluid delivery channel is an independent tube, the material of the tube is not particularly limited, and a tube material used for catheter and endoscope can be used. For example, polyurethane, polyester, polyolefin, fluorine-based resin (e.g., Teflon (registered trade mark)), nylon, silicone rubber, and the like are preferable.

The transparent fluid to be injected from the fluid delivery channel may be a gas or liquid, depending on the situation and environment under which an observation target site is placed, and a combination with an obstacle.

For example, for application to the inside of a suspended sewer piping, the transparent fluid may be water. For intravascular application, the transparent fluid may be physiological saline or, in a preferable embodiment, a predetermined amount of the patient's own blood plasma or blood serum taken in advance is used.

As the injection flow of the transparent fluid, an amount that enables rapid observation can be appropriately determined according to the outer diameter of the catheter, inner diameter of the hood, viscosity of an opaque fluid to be excluded and the like.

When the catheter is intravascularly applied and physiological saline is injected as a transparent fluid in the hood with an inner diameter of about 5 mm-4 mm, an injection time of the physiological saline is preferably about 0.1 [second]-0.3 [second] when it is synchronized with the timing when the blood flow stops. The flow rate of physiological saline is preferably about 2 [mL/second]-5 [mL/second], and can be about 1 [mL/second]-3 [mL/second] in view of the stay action in the hood tip.

The imaging channel may be an image guide that transmits images obtained at the tip to the proximal part through an optical fiber bundle, a constitution wherein a CCD camera is disposed at the tip and the image data is transmitted to the proximal part through a signal line and the like, or a constitution wherein the image data is transmitted wirelessly, and received by a receiver set outside the body.

To perform imaging at the tip more preferably, the catheter is preferably provided with a lighting channel. The lighting channel may be an embodiment of a light guide wherein the light is transmitted from the proximal part to the tip through an optical fiber, or an embodiment wherein a light emitting element such as LED and the like is disposed at the tip and light emission is operated at the proximal part through an electric cable and the like and the like, and the type of light source is not limited.

As shown in FIG. 1, the tubular body is provided with a bending mechanism that bends the tip from a linear state toward at least one lateral direction, so that an oblique opening of the hood will come close to an observation target site.

The bending mechanism is preferably a mechanism that can bend a tubular body from a linear state toward one lateral direction and permits the tubular body to return to the original linear state. Depending on the use, a mechanism capable of bi-directional bending and stretching motion is preferable, which includes returning from the aforementioned one lateral direction to the original linear state, and further, a similar bending and stretching motion to the opposite side. The aforementioned bending to the opposite side enables covering of an observation target site with a hood, corresponding to the bending of the blood vessel.

Examples of the bending mechanism include a mechanism wherein a wire is disposed under the surface layer of the main body of the tubular body from the proximal part to the tip part along the longitudinal direction (constituted such that the tubular body can be bent at a predetermined part of the tip part by pulling the wire at the proximal part), a mechanism wherein the bending motion and restoring motion of a shape-memory alloy and a heater are appropriately combined (return spring is also used as necessary), a mechanism wherein an extremely compact air-cylinder is driven by compressed air, a mechanism using a polymer actuator, what is called an “artificial muscle”, and the like. The above-mentioned bi-directional bending and stretching motion can be achieved by, for example, placing a wire at respective positions 180 degrees opposite from each other on the outer circumference of the main body of the tubular body.

As for these bending mechanisms, various known techniques of catheter and endoscope can be referred to.

In an embodiment wherein the tubular body is a simple tube and an endoscope is inserted therein, the bending mechanism equipped to the endoscope may also be utilized.

The basic shape of the hood may be a cylindrical shape as shown in FIG. 1, or a hollow circular truncated cone shape enlarging toward the tip as shown in FIG. 3 (what is called a megaphone shape), wherein the tip of its body only needs to be obliquely cut, and the obliquely-cut opening is the opening of the tip side of the hood, as shown in FIG. 1.

The basic shape of the hood may be not only literally a cylindrical shape wherein the outer diameter and the inner diameter are constant along the central axis direction and literally a circular truncated cone shape wherein the outer diameter and the inner diameter increase linearly, but also may be added with a topical part having a different outer diameter and a different inner diameter as long as the object of the present invention can be achieved. Therefore, while the basic shape of the opening at the hood tip is also a simple ellipse obtained by obliquely cutting a cylindrical shape or circular truncated cone shape, it may be a cross sectional shape corresponding to the modification applied to the shape of the hood.

The [shape obtained by obliquely cutting] in the present invention expresses the resulting shape, and it may not be necessarily formed by obliquely cutting, but formed by using a mold having such shape.

The obliquely-cut plane may be a flat plane, or a surface curved along the surface of an observation target site.

In the present invention, as shown in FIG. 1 and FIG. 3, a hood tip 22 is bent to the inside of the hood, thereby disturbing the flow of the transparent fluid f to let the fluid stay. The shape of a part adjacent to the hood tip is also preferably changed as appropriate so that the face of the tip of the hood can be a flat plane, or a surface curved along the surface of an observation target site, without undulating, even when the hood tip is bent to the inside of the hood.

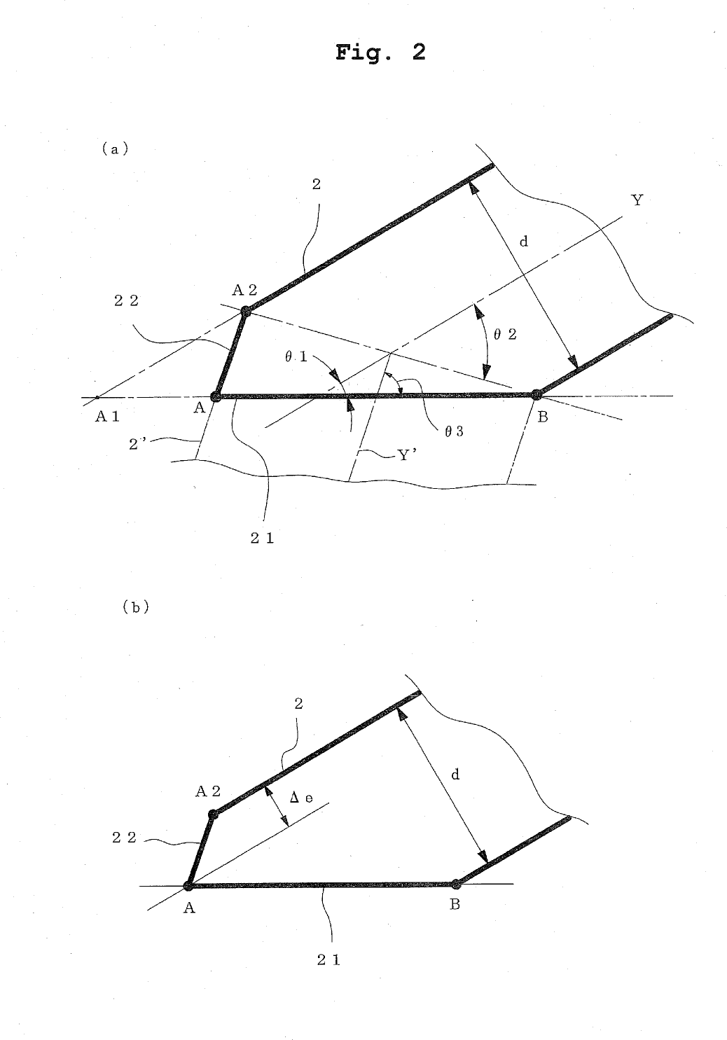

While the bending of the hood tip may be a topical bending of only that part, preferably as shown in FIG. 2, a shape obtained by bending the whole tube of the hood and cutting the tube obliquely at an end surface 21 so that it will not swell to the outside of the outer diameter of the tubular body.

In the embodiment of FIG. 2, a circular cylinder of the hood 2 having the central axis Y bends to the second circular cylinder 2′ having the central axis Y′, wherein A is a point located at the tip side of the outer periphery of the opening at the tip of the hood and B is a point located at a rear end side thereof, A2 is a point at which the hood tip starts to bend, and a [flat plane perpendicular to the plane of the paper including line segment A2-B] is the boundary plane of the bending. This bend may be a sudden folding as in the Figure or a curve forming a curvature. A1 in this Figure is a fictitious point at the tip when the hood tip is not bent.

In the embodiment of FIG. 2, the tip face 21 is a plane obtained by cutting a second circular cylinder 2′ at a [flat plane (tip face) perpendicular to the plane of the paper including line segment A-B]. This prevents point B from deviating from the outer diameter of the circular cylinder of the original hood 2. In other words, point B is on the body of the original hood.

Examples of preferable size of each part are given by referring to the embodiment of FIG. 2. Where necessary, chamfer and roundness may be formed as appropriate in narrow parts.

An angle (angle formed by central axis Y and tip face 261) θ1 of the [shape obtained by obliquely cutting] at the tip of the hood is preferably 30 degrees-60 degrees, more preferably 40 degrees-50 degrees. However, angle θ1 does not necessarily mean that one most preferably value exists, and each angle has its advantage. For example, when θ1 is as sharp as about 30 degrees, the hood advantageously covers an observation object part in the lateral direction widely by only bending a tubular body by 30 degrees. On the other hand, when θ1 is about 60 degrees, the tubular body of the hood needs to be bent up to 60 degrees to cover an observation target site in the lateral direction; however, the observation target site can be advantageously observed in a state closer to the straight gaze.

Angle θ2 formed by the boundary plane of bend [flat plane including line segment A2-B and perpendicular to paper plane] and the central axis Y determines where to start the bending, and about 50 degrees-70 degrees is preferable, and 55 degrees-65 degrees is more preferable.

Angle θ3 formed by the bent central axis Y′ and tip face 21 [flat plane including line segment A-B and perpendicular to paper plane] determines the level of bending (inclination of hood tip 22), about 65 degrees-85 degrees is preferable, and 70 degrees-80 degrees is more preferable.

As a result of the bending of hood tip 22, point A at the tip of the outer periphery of the opening of the tip of the hood protrudes into the hood. As shown in FIG. 2(b), since a protrusion amount Δe thereof is in a strong relationship with the level of disturbance of the flow of the fluid from the fluid delivery channel, for example, the amount Δe can be appropriately determined by changing the position of point A2 so that a preferable stay can be obtained.

The proportion of Δe in the inner diameter of the hood is preferably about 10%-20%. More specifically, when the inner diameter of the hood is about 4 mm-5 mm, the protrusion amount Δe is, for example, preferably about 0.4 mm-1.0 mm, more preferably about 0.6 mm-0.9 mm.

The difference between the outer diameter of the hood and the outer diameter of the tubular body is preferably small.

The hood and the tubular body may be integrated or parts separate from each other.

When the hood and the tubular body are formed as parts separate from each other, known connecting or jointing methods such as utilization of adhesive, heat seal, welding, screw-in joint, fitting, press, screw clamp, coupling and the like is appropriately used or combined to connect them.

FIG. 3(a) is a cross-sectional view showing one embodiment wherein the opening of the hood is enlarged in a flared shape (circular truncated cone shape). By enlarging the hood, the field of view is widened, and a more preferable observation becomes possible. Also in this case, the hood tip 22 is bent inward, making an injected fluid to stay.

When the hood is deformed in this manner, for example, it is preferable to impart elasticity or stretchability to the hood material, so that the hood will be shrunk upon puncture into a blood vessel and the like and inflated after entry into the blood vessel.

FIG. 3 shows another preferable embodiment of the present invention. In the example in this Figure, the basic shape of the hood is a hollow circular truncated cone shape enlarging toward the tip.

As shown in this Figure, the tubular body 1 is narrower at tip part 1a than the body outer diameter at the proximal part—intermediate part, and a hood 2 extends enlarging forward while enlarging from the outer periphery of the tip thereof.

The amount of enlarging of the hood in this case is not particularly limited and, in view of the manner of insertion into the object, the maximum outer diameter of the tip is the same as the outer diameter of the body from the proximal part to the intermediate part of the tubular body in a preferable embodiment as in FIG. 3.

Angle θ4 of expansion of the hood is preferably 25 degrees-45 degrees, more preferably 30 degrees-40 degrees.

In embodiment of FIG. 3, an endoscope with an imaging channel 12a is inserted in the inside of the tubular body 1. In FIG. 3(b), (c) showing the X-X cross-section of FIG. 3(a), a narrow part 1a of the tip of the tubular body becomes narrow such that it holds the body of the aforementioned endoscope 12a while ensuring at least a fluid delivery channel 11.

In the embodiments of FIG. 3(b), (c), while a fluid delivery channel is a gap between the tubular body 1 and the endoscope 12a, an individual tube may also be inserted. In both embodiments, the tubular body 1 is narrow such that it holds the outer circumference of the body of the endoscope 12a at 3 points at an equal distance, and the 3 parts between the 3 points are the fluid delivery channels 11.

The cross-sectional area and cross-sectional shape of each of the fluid delivery channel can be appropriately determined in consideration of the flow rate of the fluid to be discharged.

The material of the hood may be any as long as it has mechanical strength, chemical resistance, environmental resistance, corrosion resistance, biocompatibility, flexibility, stretchability and the like according to the object, such as inorganic materials (e.g., metal, ceramics and the like), organic polymer materials (e.g., plastic, silicone rubber, and the like), and the like.

Metals such as titanium, stainless steel and the like are superior in mechanical strength. For example, when the outer diameter of the hood is 4 mm-6 mm, the thickness can be 0.3 mm-0.5 mm.

As the organic polymer material, polyolefin, polyamide elastomer and the like are preferable materials from the aspects of flexibility and pressure resistance.

While organic polymer materials such as polyolefin, polyamide elastomer and the like are soft and less often injure an observation target site, when the outer diameter of the hood is 4 mm-6 mm, the thickness is preferably about 0.5 mm-0.8 mm.

When the catheter is inserted into an object such as the living organism and the like, the hood tip of the catheter may be simultaneously monitored by blood vessel angiography.

In this case, a metal marker is preferably provided on the hood so that images of the blood vessel angiography display the hood even when it is a polymer.

Examples of a preferable metal material marker include stainless steel, titanium and the like.

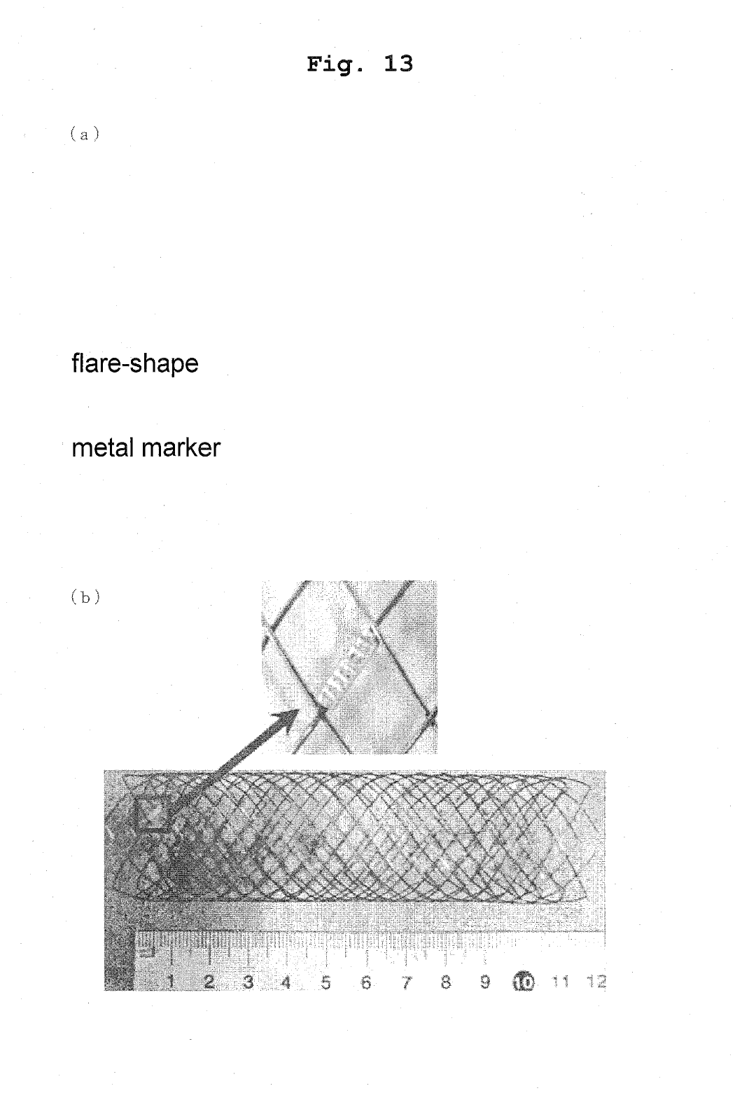

Examples of the method for forming a metal marker on the hood include a method including embedding a stainless steel wire with an extra fine diameter in the hood along the longitudinal direction thereof as shown in FIG. 13(a), and the like.

The relationship between the bending direction by the bending mechanism of the tubular body, and the opening of the hood is as follows.

As shown in FIG. 2, the bending direction and the inclination of the hood opening are related such that the tubular body bends toward the direction that brings a line segment A-B closer to parallel to the central axis Y of the tubular body of the catheter (i.e., bending direction shown in FIG. 1) or a direction opposite therefrom, wherein A is a point located at the tip side of the outer periphery (end portion including hood mass) of the opening at the tip of the hood and B is a point located at a rear end side thereof.

In actual assembly of the catheter, the direction of the opening of the hood only needs to be set in the bending direction of the bending mechanism of the tubular body.

The relationship between the configuration of each channel in the tubular body, and the inclination of the hood is preferably determined as appropriate in the bending state of the tubular body in consideration of the following actions.

(i) A fluid delivery channel is set at a position that permits more effective exclusion of an opaque fluid (blood and the like) when a transparent fluid is discharged from the fluid delivery channel.

(ii) An imaging channel is provided at a position more advantageous for imaging such as more preferable imaging of observation target site and the like.

(iii) When a lighting channel is formed, it is provided at a position more advantageous for lighting such as more preferable irradiation of light to an observation target site and the like.

(iv) As others, for example, when a forceps channel is formed, it is set at a position that ensures a space sufficient for the operation of the forceps according to the shape of the forceps to be used.

In the example of FIG. 1, a fluid delivery channel 11 is formed in the outside of the bend (upper side of Figure). This intends more effective removal of the blood that enters from the upstream side of the blood flow (left side of Figure) in the hood, and effectively hitting transparent fluid f discharged from the fluid delivery channel against the bend of the hood tip to make it stay there.

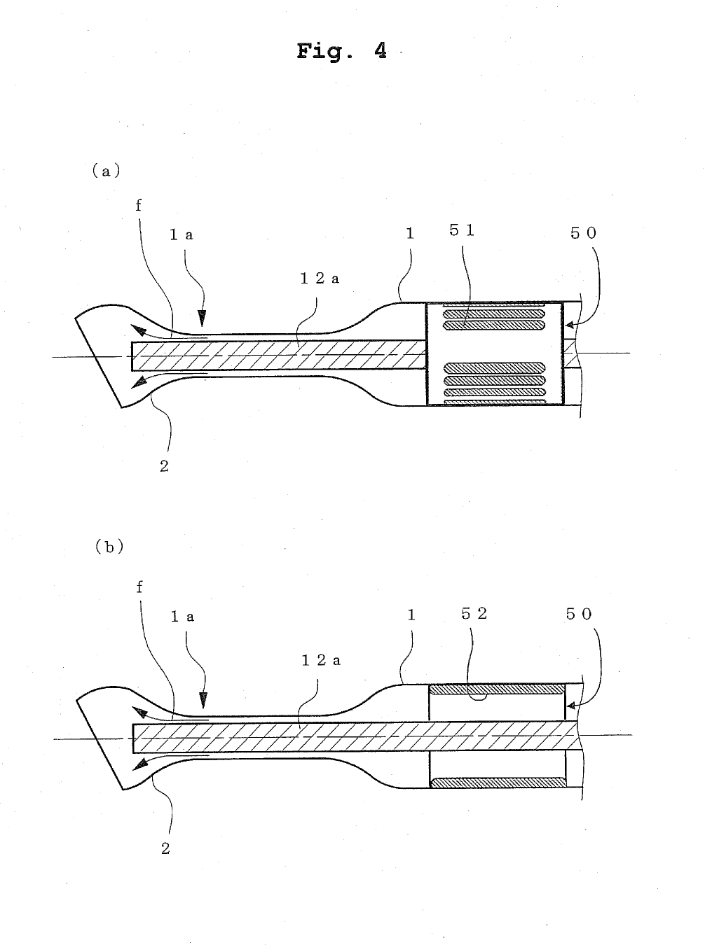

In the present invention, it is proposed to mount an ultrasonic oscillator on the body of all catheters capable of observing ahead, inter alia, catheters capable of injecting a transparent fluid and imaging around the tip, and impart an ultrasonic diagnosis function of wall surface. FIG. 4 shows one example of a specific embodiment thereof.

Ultrasonic oscillator is also called an ultrasonic transducer, and functions as an ultrasonication transmitting-receiving element in combination with an outside driving circuit. Ultrasonic diagnosis, particularly, intravascular ultrasound (IVUS), using an ultrasonic oscillator can obtain tomographic images of the inner wall over 360 degrees from the inside of the blood vessel.

The techniques per se for performing an ultrasonic diagnosis using an ultrasonic oscillator (material and structure of the oscillator, configuration of electric and electronic circuits for driving elements and transmitting and receiving ultrasonic waves, image display device and the like), and the techniques per se for applying same for an intravascular ultrasonic diagnosis are well known as described in, for example, the above-mentioned patent documents 4, 5 and the like.

However, since the equipment for intravascular ultrasonic diagnosis used conventionally simply mounts an ultrasonic oscillator on the body of a long and narrow equipment, such as catheter, it is defective in that it cannot obtain images ahead, though it can obtain images of the blood vessel wall.

Therefore, when ultrasonic diagnosis of blood vessel inner wall is actually performed by inserting an equipment for intravascular ultrasonic diagnosis and advancing the equipment to the object site, the equipment needs to be advanced depending solely on the feeling at the operation side, and an accident of breaking through the blood vessel wall with the tip of the equipment sometimes occurs.

In contrast, by mounting an ultrasonic oscillator 51 on the body of the catheter capable of imaging the blood vessel near the tip while injecting a transparent fluid as in FIG. 4(a), an ultrasonic diagnosis of the wall surface is enabled and the vicinity of the tip can be confirmed with images, and an accident of breaking through the blood vessel wall with the tip can be prevented.

In addition, by mounting an ultrasonic oscillator for ultrasonic diagnosis on the body of the catheter of the present invention, lesions in the inside of the blood vessel wall (for example, atherosclerosis, calcification and the like) and elasticity property of the blood vessel (level of arteriosclerosis) can be known.

In the embodiment of FIG. 4(a), a plurality of ultrasonic oscillators 51 are disposed at regular intervals to surround the main body of the tubular body in the circumferential direction to form an electronic phased array 50. While the ultrasonic oscillators 51 may be exposed outside, they are covered with a tube forming the tubular body in the embodiment of FIG. 4(a) so that the inner wall of the blood vessel will not be injured.

The embodiment of FIG. 4(a) is equipped with a drive device, a control device, an image display device and the like for the oscillator, and constituted to be able to perform a phased array method (not shown). Known techniques can be referred to as for these devices themselves.

As shown in the inner cross section of FIG. 4(b), an endoscope 12a passes a through-hole 52 in the central part of the electronic phased array 50, and a transparent fluid (e.g., physiological saline) f can head for the tip through the space.

In the present invention, when an ultrasonic oscillator for intravascular ultrasonic diagnosis is formed on the main body of the tubular body, an ultrasonic oscillator may be mechanically rotated along the periphery of the main body of the tubular body. However, the whole size becomes thick due to such rotation mechanism. As shown in FIG. 4(b), therefore, an electronic phased array wherein many elements are fixedly dispose is a preferable embodiment.

A channel for feeding a transparent fluid (e.g., physiological saline) may be formed in the inside of an endoscope 12a, the endoscope becomes thick. In contrast, an embodiment utilizing a space along the outside of the endoscope as shown in FIG. 4(b) is preferable since the outer diameter becomes compact.

Next, the constitution of an intravascular observation system using the catheter according to the present invention is explained.

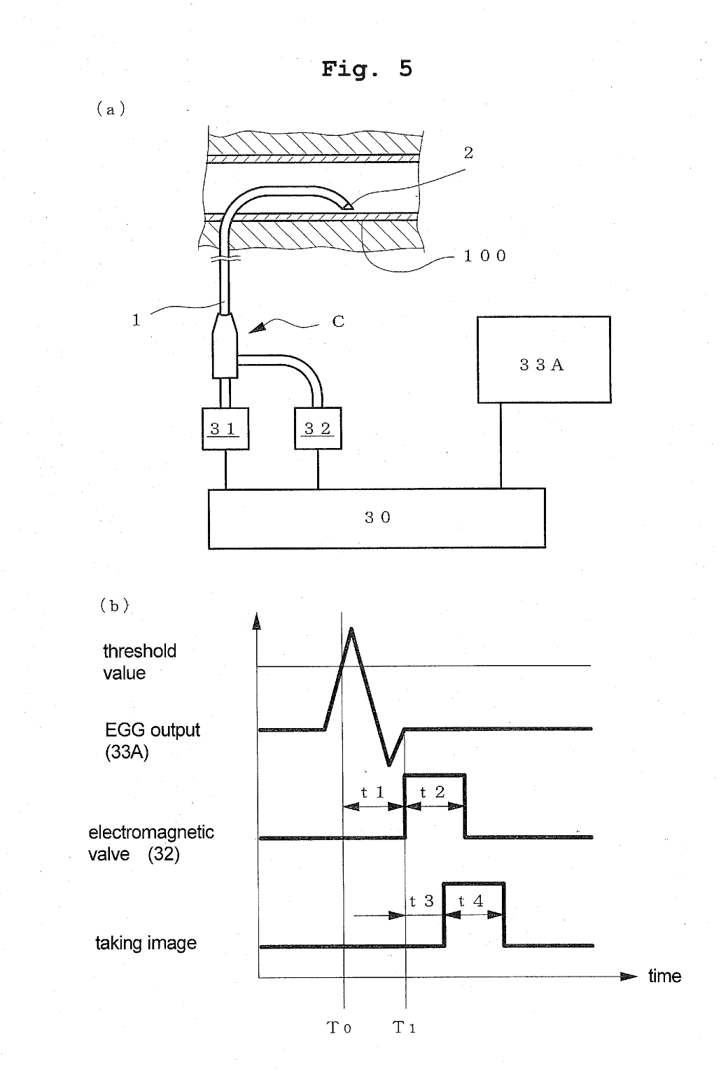

This system is composed of at least the catheter C according to the present invention, a fluid delivery device 32, and a control device 30, as one embodiment of the constitution is schematically shown in FIG. 5(a). The fluid delivery device 32 is a pump device for sending a transparent fluid to a fluid delivery channel of the tubular body 1 of the catheter C, and is constituted such that the delivery and stop of the fluid to the fluid delivery channel can be performed according to the instruction (output signal etc.) from the control device 30.

The control device 30 is constituted such that it can receive a signal indicating the motion of the heart of a patient to be inserted with the aforementioned catheter as an input signal. In the example constitution of FIG. 5(a), catheter C is inserted into a blood vessel 100 of the patient, and an output signal of an electrocardiogram device (ECG) 33A is input in the control device 30. The electrocardiogram device 33A may be regarded a sensor instrument included in the system of the present invention, or an outside signal source that emits a signal indicating the motion of the heart of patients.

The control device 30 may be constituted mainly based on the sequence circuit. However, since a complicated signal control including monitoring and analyzing a signal indicating the motion of the heart of patients, and injecting a transparent fluid in an appropriate synchronization with the signal, an embodiment constituted mainly based on a computer, with which an operator can finely adjust the parameter of each timing on the screen or with an external control panel, is preferable.

An interface necessary for the connection of external devices such as pump, electromagnetic valve and the like and a control device, an image display device, a printer and the like can be formed as appropriate.

An important characteristic of the system is that the control device 30 is constituted to control a fluid delivery device 32 such that a signal showing the motion of the heart of patients (output signal of electrocardiogram device 33A in the example of FIG. 5(a)) is received as an input signal, calculation is performed based on the input signal, and a predetermined amount of a transparent fluid is injected at the tip of catheter C from the tip of a fluid delivery channel 11 into the inside of hood 2 at an appropriate time period when the blood flow stops.

As a result, an operator of the catheter of the present invention delivers the catheter to an observation target site, operates the bending mechanism to enclose the observation target site with the hood, and discharges an appropriate amount of physiological saline in the hood at an appropriate time period when the blood flow stops, whereby the observation target site can be seen.

The fluid delivery device 32 may be a simple pump or cylinder that starts the motion of feeding a transparent fluid by the instruction from the control device 30. To inject a transparent fluid with a steep rise in accordance with the timing of cease of the blood flow, an embodiment wherein a transparent fluid is contained in a pressure container, the inside of the container is always pressurized with a compressed air, and the transparent fluid is injected or stopped by opening or closing an electromagnetic valve formed on an injection port of the container is preferable.

In the example of FIG. 5(a), a preferable embodiment includes an imaging device 31, and images sent from an imaging channel of tubular body 1 are converted to electric signals (digital image data, analogue video signal and the like) and sent to the control device 30.

In such imaging device, a camera (photographic equipment), a video camera and the like can be appropriately set according to the imaging channel or use object. When an imaging device such as a CCD camera and the like is included in the tip of the imaging channel, the signal output thereof may be directly input in the control device 30.

FIG. 5(b) is a time chart showing the relationship between a signal from an electrocardiogram device (ECG output) indicating the motion of the heart of patients, and the timing of motion of an electromagnetic valve and the appropriate timing for taking images based on the signal, when a transparent fluid is constantly delivered by controlling an electromagnetic valve.

As shown in FIG. 5(b), from the peak portion showing the cardiac systole of the wave shapes of ECG output, an initial time T0 is created by determining an appropriate threshold value, a delay time period from the initial time to the cease of the intravascular blood flow is experimentally obtained, and the motion of the electromagnetic valve is started from time T1 after delay of an appropriate delay time period t1 from the initial time such that a transparent fluid is injected from a catheter tip at the moment when the blood flow stops. In this case, it is preferable to take into consideration a delay time from the start of the motion of electromagnetic valve to the injection of the transparent fluid. t2 is a release time period of the electromagnetic valve based on the end of the cease of the blood flow or in consideration of blood dilution of patients by dozing with the transparent fluid.

Furthermore, a delay time period t3 from the motion start time T1 of the electromagnetic valve to the transparency in the hood is experimentally obtained, and image taking is started after lapse of the delay time period t3. t4 is an imaging time period based on the time period during which the transparency in the hood is lost.

The motion of the imaging channel may be constituted, as shown in FIG. 5(b), to start imaging of an observation target site in synchronization with the timing of injection of a transparent fluid from the fluid delivery channel (after a suitable delay time t3), and perform imaging only for a predetermined time period t4.

When an imaging channel always performs imaging of an observation target site and continuously outputs image signals, a control device may start recording of images in synchronization with the timing of injection of a transparent fluid, and perform recording of images only for a predetermined time period.

### EXAMPLES

### Example 1

A circulation device that simulatively reproduces pulsation of the heart and variation in the blood flow associated therewith in the circulatory system of the human body was constituted and, using the device, the usefulness of the catheter and the system according to the present invention was confirmed.

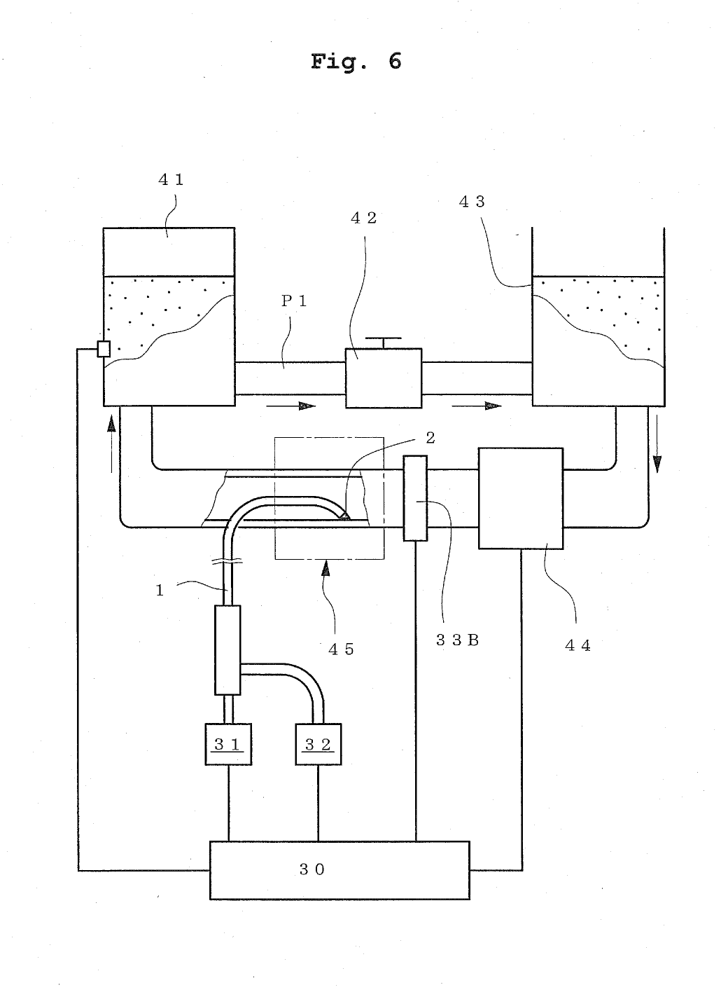

FIG. 6 is a block diagram showing the constitution of the circulation device. As shown in this Figure, the circulation device comprises a closed chamber 41 for arterial pressure loading, a resistance 42, a venous system reservoir 43 and a compressed air type pulsatile flow pump 44 simulating the systole-diastole motion of the heart, which are connected sequentially in a loop by a piping pipe P1 simulating the blood vessel, and a circulatory organ that circulates a liquid in the arrow direction while generating pulsation is reproduced.

The action of the closed chamber 41 for arterial pressure loading simulates the compliance (elasticity) of the artery that temporarily preserves the blood ejected from the cardiac ventricle (pulsatile flow pump) during cardiac systole.

The action of the venous system reservoir 43 simulates the action of the atrium that preserves the blood in order to deliver the blood at once to the cardiac ventricle during cardiac diastole.

The action of the resistance 42 simulates the resistance of the blood vessel (total peripheral circulation resistance).

The fluid that circulates in the pipe was a white turbid liquid obtained by mixing tap water with a commercially available white turbid bathing agent (main component: sodium hydrogen carbonate) at a ratio of 10 [g/L].

The operation characteristics of the above-mentioned circulation device are as follows.

frequency: 1 [Hz]

amplitude: 5 [Vp-p]

duty ratio: 30[%]

average internal pressure of closed chamber: 13.3 [kPa]

pulse pressure: 8.0 [kPa]

average flow rate: 6 [L/min]

A constitution wherein a flow meter 33B was connected immediately after the pump 44, which was the heart, to record a signal indicating the variation in the fluid flow in the pipe, and a driving signal of the pump 44 was input instead of electrocardiogram in the control device 30 of the system of the present invention.

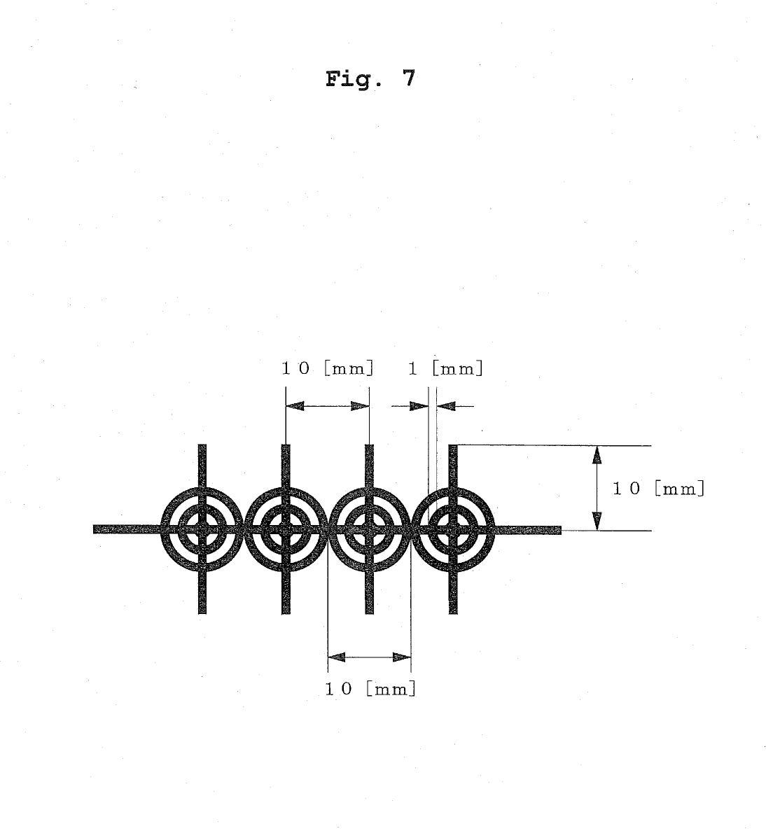

In the observation site 45 in the pipe was disposed, as a target of the observation object, a concentric circular mark shown in FIG. 7. As in this Figure, the mark is drawn with the same 1 mm-thick line and is a pattern made of 4 concentric circles each consisting of a circle with an outer diameter of 10 mm, a circle with an outer diameter of 6 mm, and a circle with an outer diameter of 2 mm, and a cross-shaped pattern in combination.

Image of this mark was taken, and how much the white turbid liquid was removed and how clearly the mark can be seen was evaluated by luminance difference. The “luminance difference” is shown in a numeric value converted with the luminance of the white part of the target as 255, and the luminance of the black part as 0 when filled with water.

The catheter system of the present invention applied to the above-mentioned circulation device is the same as that shown in FIG. 5(a). In this Example, the catheter is inserted in the piping pipe and the tip thereof has reached an observation site 45.

### [Hood]

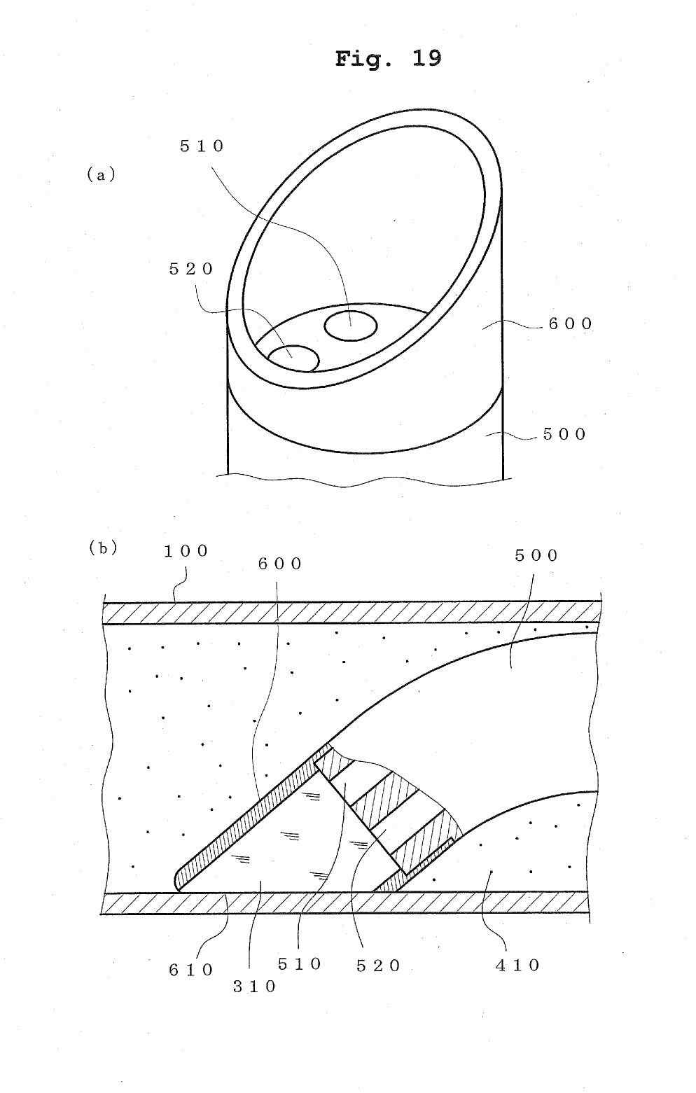

As for hood, the Example product was one having the shape shown in FIG. 2(a), and the Comparative Example product was one having the shape without a bend in the hood tip as shown in FIG. 19(b). The difference between the Example product and the Comparative Example product is only the presence or absence of a bend in the hood tip.

In the Example product and the Comparative Example product, as shown in FIG. 2(a), the basic shape of the hood was a cylindrical shape, the inner diameter was 5.5 mm, and the angle θ1 formed by the central axis Y of the cylindrical shape and the opening surface 21 was 40 degrees.

In the Example product, as shown in FIG. 2(a), angle θ2 formed by the boundary plane of the bend of the hood tip [flat plane including line segment A2-B and perpendicular to paper plane] and the central axis Y was 60 degrees, angle θ3 formed by the bent central axis Y′ and a tip face 21 [flat plane including line segment A2-B and perpendicular to paper plane] was 75 degrees.

The constitution of the fluid delivery device was that transparent water was filled in a pressure container, the inside of the container was constantly pressurized to 0.3 MPa, and water was discharged and stopped by opening an electromagnetic valve set at an injection port of the container for 300 msec (discharge time period 300 msec). The spontaneous rate of the pump was 100 times per minute.

In addition, based on the variation in the fluid flow in the pipe by a flow meter 33B, each delay time was appropriately set, and discharge was made to be performed at an observation site at the moment when the water flow stops.

### [Property Confirmation Experiment]

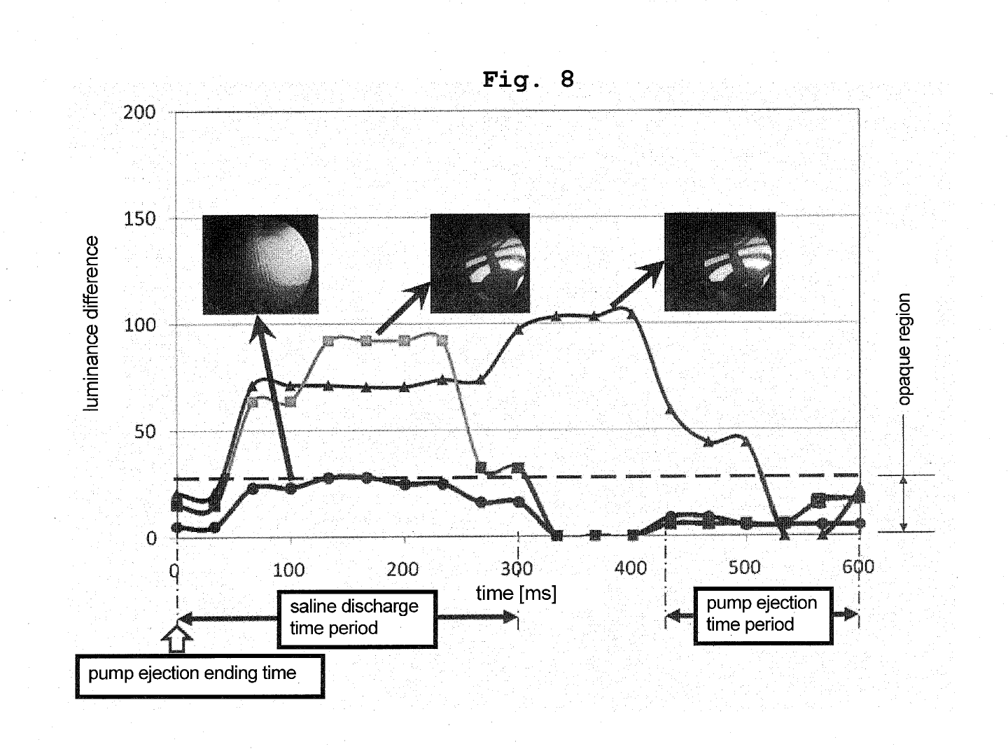

First, the tip of the hood face of the Example product catheter was maintained at about 1 mm apart from the surface of the target mark, and water was discharged. As shown in the graph chart of FIG. 8 by plotting with square-shaped points, a peak of the luminance difference permitting clear identification of the target mark could be obtained during a 300 msec discharge time period. The total discharge amount of water per one time was 4 ml. The image of the mark taken at that time period is as shown in the graph chart of FIG. 8.

In contrast, when the tip of the hood face of the Comparative Example product catheter was maintained in contact with the mark surface, and water was discharged, as shown in the graph chart of FIG. 8 by plotting with triangle-shaped points, the mark could be identified with certain clarity during a 300 msec discharge time period, but a peak of clear identification of the mark was obtained after the 300 msec discharge time period.

When the tip of the hood face of the Comparative Example product catheter was maintained at about 1 mm apart from the surface of the mark, and water was discharged, the mark could not be identified clearly as shown in the graph chart of FIG. 8 by plotting with circular-shaped points.

From the above experiment, it was found that a mark can be clearly identified even when the tip face of the hood is apart from the mark surface according to the present invention.

### Example 2

How clearly the mark could be seen by the Example product when the internal pressure of the pressure container and the open time period (discharge time period) of the electromagnetic valve in the above-mentioned Example 1 were changed was confirmed.

In this Example, the internal pressure of the pressure container was set to 0.1 MPa, and the open time period (discharge time period) of the electromagnetic valve was set to 100 msec.

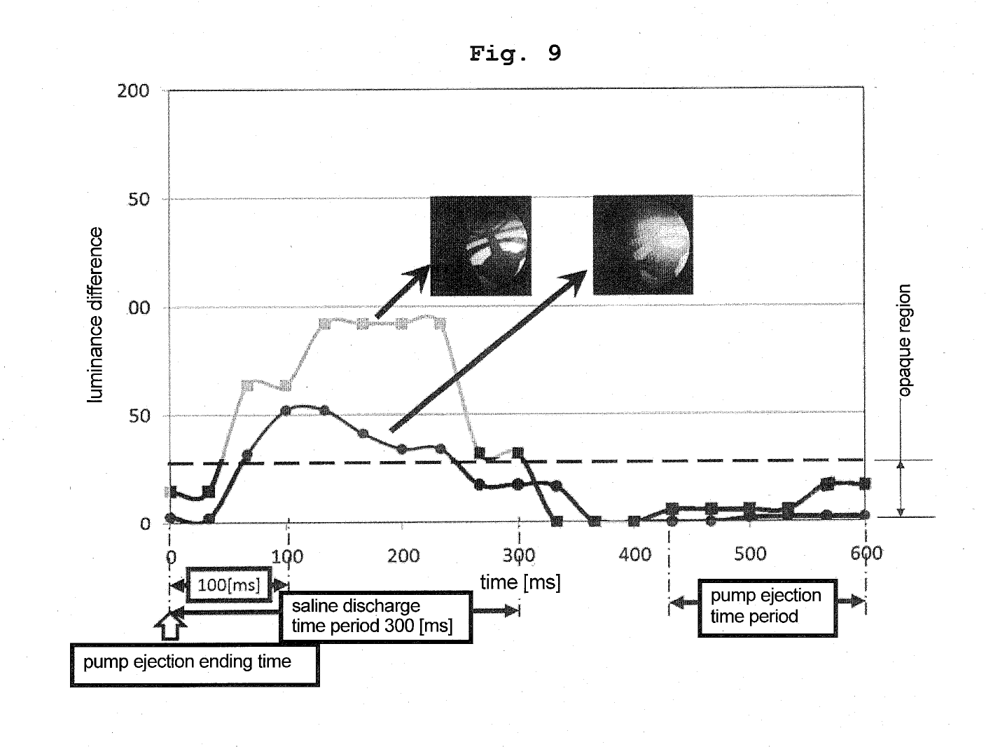

The results of the Example product in this Example (pressure 0.1 MPa, discharge time period 100 msec) are shown in the graph chart of FIG. 9 by plotting with circular-shaped points, and the image of the mark taken at that time is shown in the graph chart in this Figure.

In addition, the results of the Example product in Example 1 (pressure 0.3 MPa, discharge time period 300 msec) are shown overlapped in the same graph chart of FIG. 9 by plotting with square-shaped points.

The tip of the hood face of the Example product catheter was maintained at about 1 mm apart from the surface of the mark, and water was discharged (pressure 0.1 MPa, discharge time period 100 msec). As shown in the graph chart of FIG. 9 by plotting with circular-shaped points, a peak of clear identification of the mark could be obtained in 100 msec discharge time period.

From the results of this Example, it was found that the mark could be observed even when the discharge pressure and the discharge time were decreased. The total discharge amount of water per one time decreased to 0.6 ml since the discharge pressure and the discharge time period were decreased, which has clarified that discharge and imaging can be performed many more times in living organisms. In addition, since the mark can be clearly identified even when the tip of the hood face is maintained at about 1 mm apart from the surface of the mark, it was found that the surface of the inner wall of the blood vessel can be continuously observed by rotating the tip of the catheter in the circumferential direction.

### Example 3

In this Example, a bile duct stent (length 45 mm, diameter 10 mm, material: nickel-titanium alloy) was inserted into the descending aorta of a swine (body weight about 30 kg) under general anesthesia, and an artificial blood vessel (length 15 mm, diameter 10 mm, material: dacron base, collagen coated) was anastomosed thereto. The catheter of the present invention was inserted backwardly from the abdominal aorta, physiological saline was discharged in synchronization with the cardiac electrogram of the swine, and visualization of the stent, artificial blood vessel, blood vessel branched part and the like was examined. At that time, the position of the catheter was identified by an angiography device (blood vessel angiography device).

The catheter was made from polyurethane, wherein the tubular body was a tube having an outer diameter of 6.2 mm and an inner diameter of 5.6 mm, an endoscope (outer diameter 1.4 mm) was inserted thereinto, and a hood was joined with the tip with a silicone rubber-based adhesive.

The hood had the same outer diameter, inner diameter with the tubular body, and the shape of the tip was similar to the hood of Example 1.

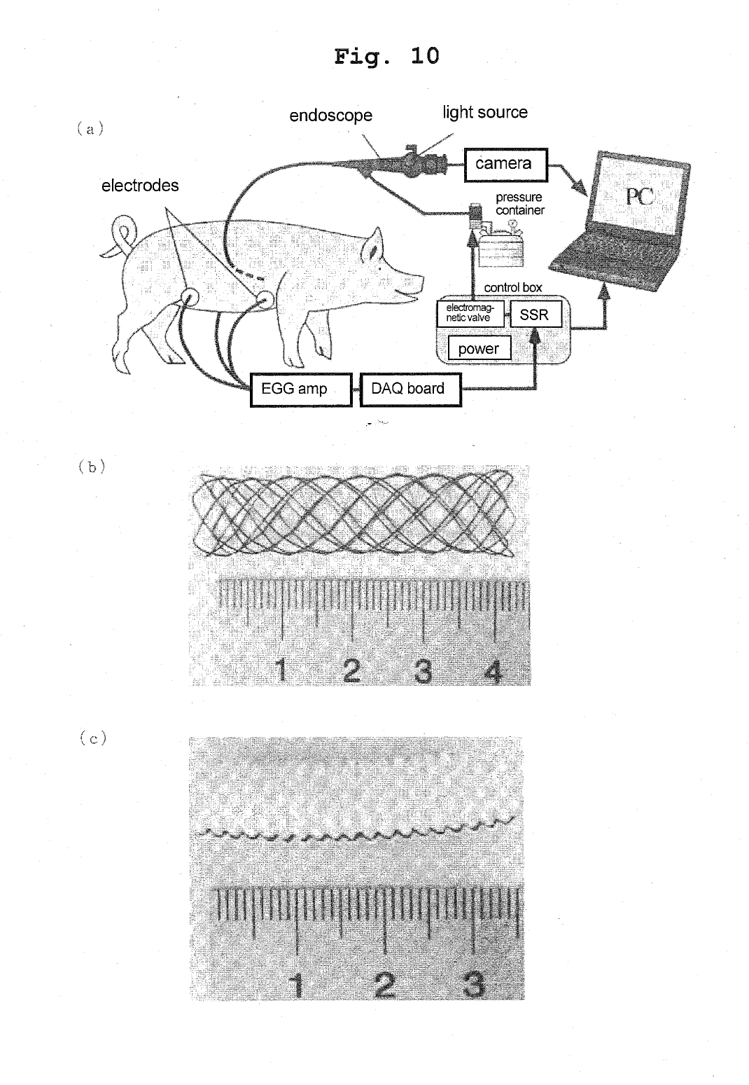

The constitution of the whole system is shown in FIG. 10(a). FIG. 10(b) is a photograph showing the stent, and FIG. 10(c) is a photograph showing the artificial blood vessel.

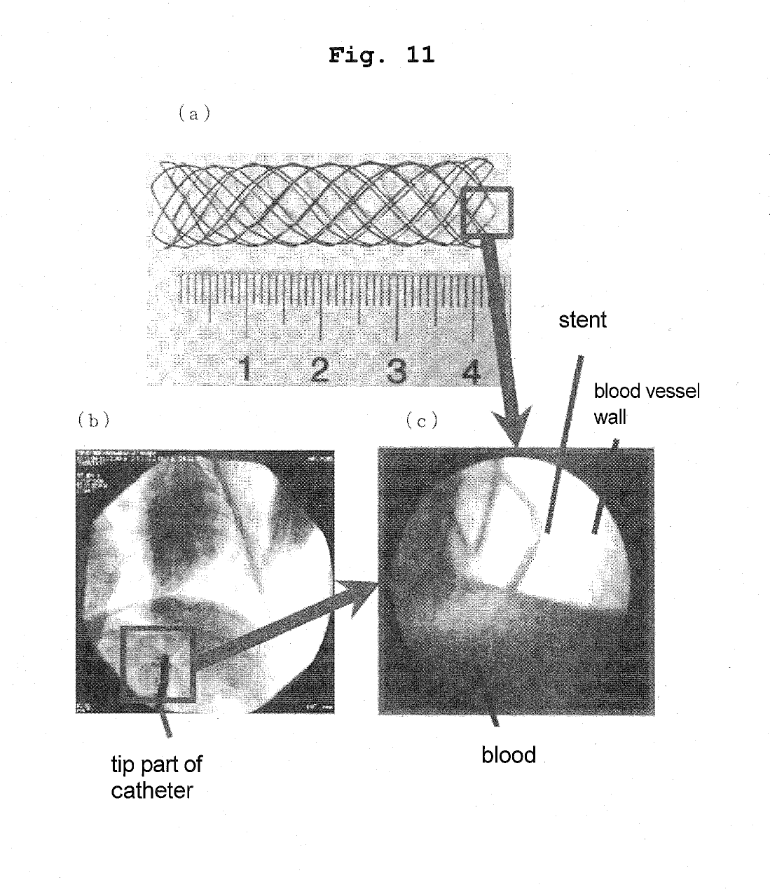

FIG. 11 shows an angioimaging photograph (FIG. 11(b)) and an endoscope imaging photograph (FIG. 11(c)), when a stent (FIG. 11(a)) was inserted.

As shown in FIG. 11(b), the tip of the catheter was confirmed in the descending aorta by angiography imaging, and indwelling of the stent can be observed. As shown in FIG. 11(c), visualization of the terminal portion of the stent in blood could be confirmed by endoscope imaging while discharging physiological saline.

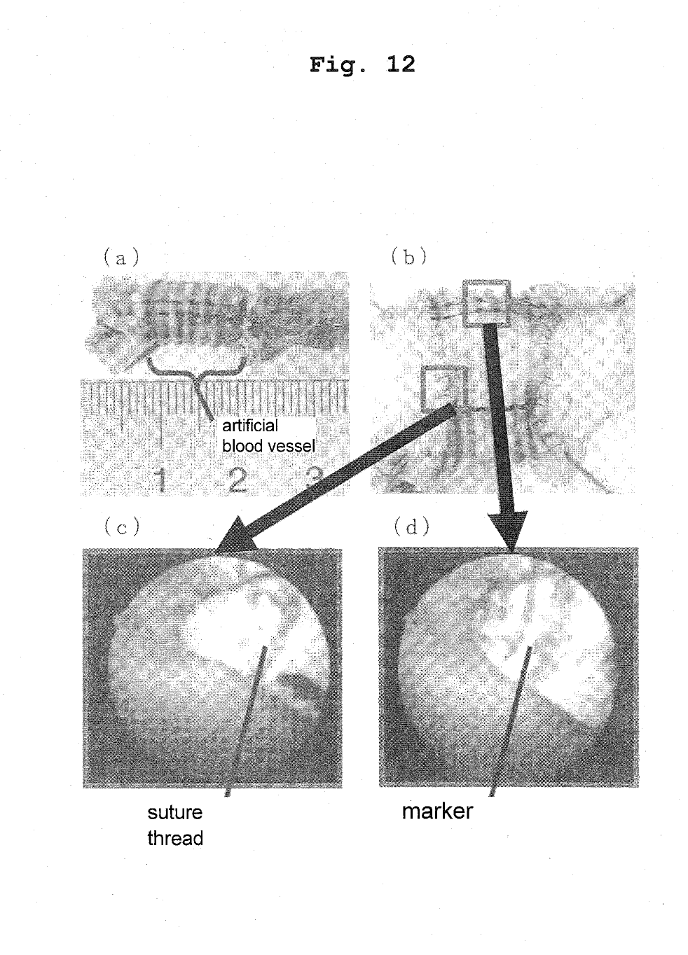

FIG. 12(c), (d) are endoscope imaging photographs when the artificial blood vessel was end-to-end anastomosed to the descending aorta. FIG. 12(a), (b) are the artificial blood vessel after this Example taken out together with the blood vessel suture part. As shown in FIG. 12(b), a suture thread is present on both ends of the artificial blood vessel, and a marker (linear mark sewn with black thread) is formed on the wall surface of the artificial blood vessel in the pipe axial direction.

As shown in the photographs of FIG. 12(c), (d), visualization of the suture thread in the anastomotic part and the marker of the artificial blood vessel could be confirmed. In actual observation experiment, the photographs of FIG. 12(c), (d) are color photographs, and the suture thread on both ends of the artificial blood vessel, and the marker in the pipe axial direction are more clearly identifiable than in the black-and-white photographs attached to the present specification.

### Example 4

In this Example, a stent (length 45 mm, diameter 10 mm, material: nickel-titanium alloy) was inserted into the thoracic aorta of a swine (body weight about 35 kg) under general anesthesia. Using a catheter having a hood with a flared shape (circular truncated cone shape) as shown in FIG. 13(a) attached to the tip, as shown in FIG. 13(b), one of the meshes of the net at the terminal portion of the stent was enlarged to be a mark, and observation of the mark portion was tried. The conditions other than the hood of the catheter are the same as in Example 3.

The material of the hood was silicone rubber, and the spread angle was 35 degrees. Angle θ1 formed by the cylindrical-shaped central axis Y and the opening surface 21 was set to 30 degrees.

The aforementioned catheter was inserted from the abdominal artery, physiological saline was discharged in synchronization with the cardiac electrogram of the swine while confirming the visualized site by an angiography device, the aforementioned mark of the stent was observed.

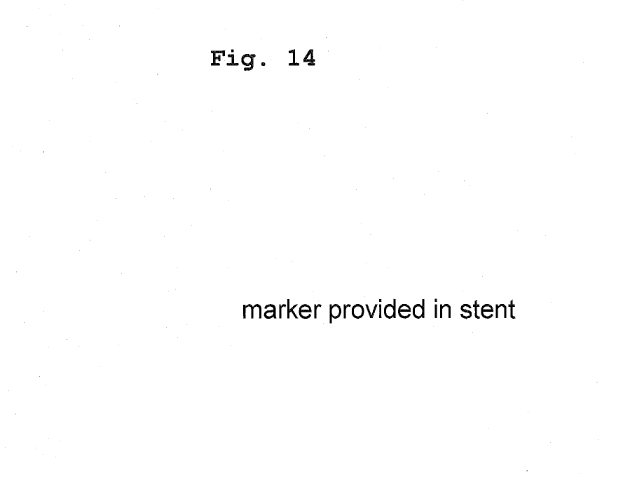

As shown in the endoscope imaging photograph of FIG. 14, visualization of the aforementioned mark formed on the stent could be confirmed even in blood.

### Example 5

With the aim of improving the practicality and broad utility of the catheter and the intravascular observation system according to the present invention, a commercially available endoscope is inserted into an outer tube produced tentatively and used as the catheter of the present invention. A large blood vessel endoscope system was constituted, and a simulative basic characteristics test was performed in the same manner as in Example 1.

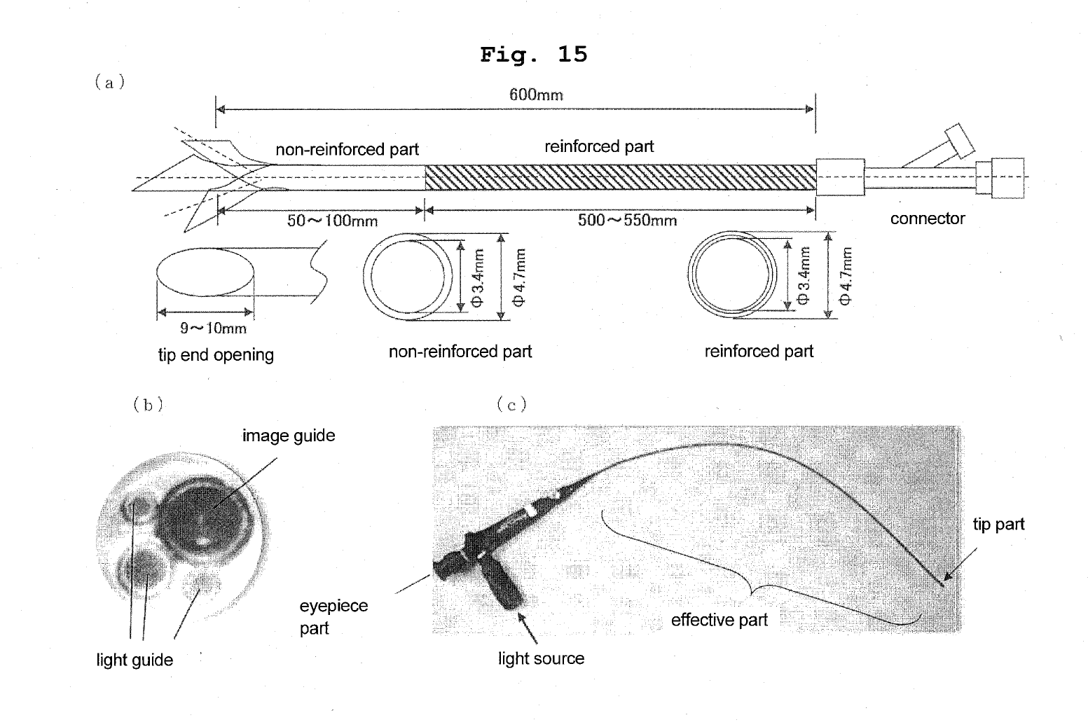

The catheter produced in this Example is the outer tube shown in FIG. 15(a) inserted with a commercially available flexible endoscope, which is a simplified constitution for performing intravascular visualization by injecting physiological saline from a connection port provided at the proximal side and discharging same from the tip. The physiological saline is discharged through a space between an outer tube and the catheter into the hood at the tip, as shown in FIG. 3(a).

The commercially available flexible endoscope has a tip face shown in FIG. 15(b), and the whole is as shown in FIG. 15(c).

The above-mentioned outer tube is largely divided into a connector part at the proximal side, an intermediate reinforced part, and a non-reinforced part on the tip side, as shown in FIG. 15(a). An endoscope insertion port and a physiological saline connection port are formed in the connector part. In an intermediate reinforced part, to increase pressure resistance and kink resistance, a tubular braid formed by braiding a metal element wire is set beneath the outermost covering layer of the outer tube. The material of the element wire of the braid is stainless steel (medical reinforcing material such as titanium and the like is also possible), which is so-called a rectangular wire (thickness 0.04 mm×width 0.11 mm). The braiding pitch of the braid is 2.8 mm, and the outer diameter of the braid is 3.66 mm. The above-mentioned commercially available flexible endoscope passes through the inside of the tubular braid.

The reinforced part on the tip has a flexible structure free of the aforementioned braid so that it can bend by the head swing of the endoscope as shown in FIG. 15(a), and a hood is integrally formed on the tip. The hood has the shape shown in FIG. 3(a).

The outer diameter of the non-reinforced part (excluding spread of the hood) and the reinforced part is both 4.8 mm, which is the size enabling an approach from the femoral artery of adult human.

As the material of the outer most covering layer of the outer tube, different polyamide resins are used for the non-reinforced part and the reinforced part. The non-reinforced part was made flexible by using a material having Shore D hardness of 25 as defined in JIS K6253, and a material having Shore D hardness of 40 (in a state without reinforcement by the above-mentioned tubular braid) was used for the reinforced part.

The constitutions of the external control device and piping for discharging physiological saline from the catheter and imaging are the same as those shown in Example 3 and FIG. 10(a), and the outline is as follows. A pressure container filled with physiological saline is pressurized by a gas cylinder and, in synchronization with the ECG waveform from the outside, sent to the catheter by opening and closing of the electromagnetic valve, and discharged from the tip. It is also possible to display only the visualized part during discharge on a monitor screen by processing the movie, obtained by an image guide, by a computer program.

As in FIG. 10(a), a separate control box was provided, and the constitution permits easy fine adjustment of the setting of the discharge time period (electromagnetic valve opening and closing time period), timing (electromagnetic valve opening and closing delay time period), image taking time period and image taking delay time period shown in FIG. 5(b) by an actual operation of a variable resistor.

Using the system constituted as mentioned above and the circulation device of FIG. 6 explained in the above-mentioned Example 1, and by operation procedures in the same manner as in Example 1, a property confirmation experiment was performed.

### [Static Characteristic Test]

A pressure container charged with water instead of physiological saline was pressurized by a gas cylinder, and water was discharged by changing the internal pressure by 0.1 MPa between 0.1-0.4 MPa, and each flow rate [ml/min] was measured.

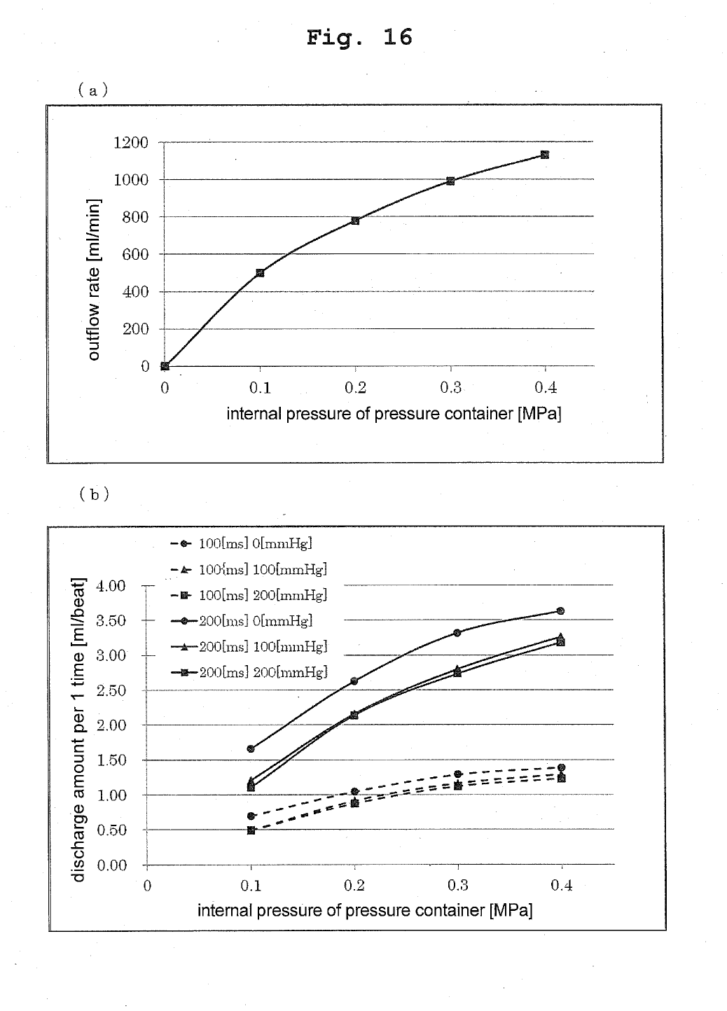

The experiment results are shown in the graph of FIG. 16(a). From the obtained graph, it can be confirmed that the internal pressure of the pressure container and the discharge amount are almost proportional.

### [Dynamic Characteristic Test]

As shown in FIG. 6, the tip of the catheter was inserted in a piping pipe P1, a closed chamber for arterial pressure loading was operated, and a load pressure (0 MPa, 0.0133 MPa (=100 mmHg), 0.0266 MPa (=200 mmHg) simulating the arterial pressure was applied to the discharge port in the tip of the catheter. Then, the internal pressure of the pressure container was changed between 0.1-0.4 MPa and one discharge time period was changed between 100-200 ms. The discharge amount [ml/beat] per one time was measured for each case.

The experiment results are shown in the graph of FIG. 16(b). It could be confirmed from the graph of FIG. 16(b) that the discharge amount can be controlled to a desired value by changing the internal pressure of the pressure container and the discharge time period, and the value is almost free of an influence of the pressure on the discharge port, namely, arterial pressure.

### [In Vitro Visualizing Performance Evaluation Experiment]

In the same manner as in Example 1 and using the circulation circuit of FIG. 6, an in vitro visualizing performance evaluation experiment was performed. As a circulation fluid, a white turbid liquid as in Example 1 was used as a substitute of blood, and the mark of FIG. 7 placed on the tube wall inner surface (corresponding to aorta wall inner surface) was observed. As the trigger signal for discharge, an artificial ECG waveform signal was input. At this time, the circulation circuit was driven assuming the physiological conditions of the experiment animal (swine).

The discharge time period was changed between 100-200 msec and the internal pressure of the pressure container was changed between 0.1-0.2 MPa, and the mark was imaged while keeping the opening of the tip of the hood in a non-contact state by holding same at about 1 mm apart from the mark surface. In the same manner as in Example 1, and how much the white turbid liquid was removed and how clearly the mark can be seen was evaluated by luminance difference.

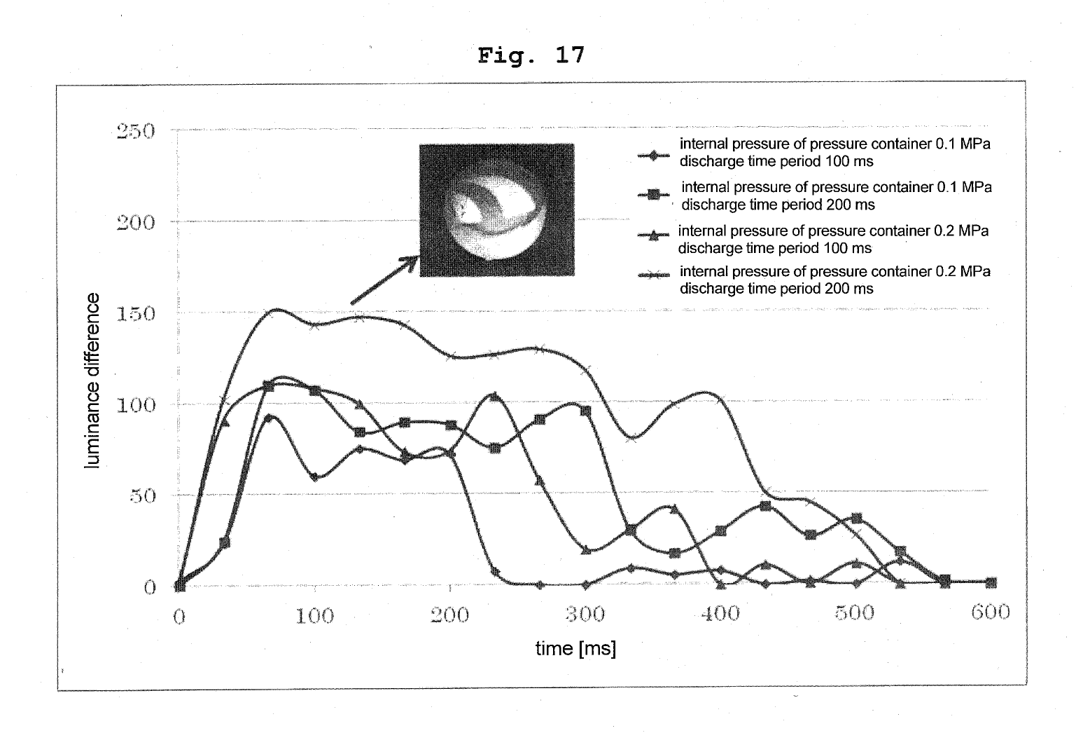

The experiment results are shown in the graph of FIG. 17. From the graph in this Figure, it can be confirmed that the mark can be seen under respective discharge conditions. In addition, in all conditions, the visualization lasted longer than the discharge time period of physiological saline. This is because the discharged physiological saline stays in the hood.

### INDUSTRIAL APPLICABILITY

Using the catheter and the system of the present invention in a suspended opaque fluid such as in a blood vessel, an opaque fluid in the periphery of an observation target site can be effectively removed by dispensing a small amount of a transparent fluid even when an end surface of the hood is about 0.5 mm-1 mm apart from the observation target site, and the observation target site can be observed visually.

This application is based on a patent application No. 2011-080732 filed in Japan (filing date: Mar. 31, 2011), the contents of which are incorporated in full herein.

1-13. (canceled)

14. A catheter having an endoscope function, comprising:
a tubular body, and
a hood extending forward from the outer periphery of the tip of the tubular body, wherein
the aforementioned tubular body comprises at least a fluid delivery channel for injecting forward a fluid from the tip of the tubular body, and an imaging channel for observation of the external environment from the tip of the tubular body, and a bending mechanism capable of bending a section with a predetermined length from the tip of the tubular body toward at least one lateral direction is provided,
the aforementioned hood has a basic shape of a cylindrical shape, or a hollow circular truncated cone shape enlarging toward the tip, the tip of the hood has a shape obtained by obliquely-cutting the aforementioned basic shape, the hollow in the hood opens at the tip face thereof and, in addition thereto, in the wall part of the outer periphery of the tip of the hood, at least a part located at the tip is bent toward the inside of the hood to prevent the flow of the aforementioned fluid heading toward the outside from a part located at the tip of the aforementioned opening.

15. The catheter according to claim 14, wherein the basic shape of the hood tip is a shape obtained by cutting the cylindrical shape or circular truncated cone shape, which is the basic shape of the hood, with a flat plane forming an angle of 30 degrees-60 degrees to the central axis thereof.

16. The catheter according to claim 14, wherein the basic shape of the hood is a cylindrical shape having the same outer diameter as that of the tubular body.

17. The catheter according to claim 14, wherein the basic shape of the hood is a hollow circular truncated cone shape enlarging toward the tip,
the outer diameter of the tip of the tubular body is narrower than the outer diameter of the body from the proximal part to an intermediate part of the tubular body, and the hood extends enlarging forward from the outer periphery of the tip,
the maximum outer diameter of the tip of the hood is the same as the outer diameter of the body from the proximal part to the intermediate part of the tubular body.

18. The catheter according to claim 17, wherein an endoscope with an imaging channel is inserted in the inside of the tubular body of the catheter, a narrow part of the tip of the tubular body secures at least a fluid delivery channel and holds the body of the aforementioned endoscope.

19. The catheter according to claim 14, wherein the bending direction by the bending mechanism and the direction of inclination of the hood opening are related such that the tip of the tubular body part bends toward the direction that brings a line segment connecting A and B closer to parallel to the central axis of the catheter or a direction opposite therefrom, wherein A is a point located at the tip side of the outer periphery of the opening at the tip of the hood and B is a point located at a rear end side thereof.

20. The catheter according to claim 14, further comprising one or more ultrasonic oscillators, which act as ultrasonic wave transmitting-receiving elements, on the tubular body of the catheter, to enable an ultrasonic diagnosis of the observation object inner wall for which the catheter is inserted.

21. The catheter according to claim 20, wherein a plurality of ultrasonic oscillators are provided as an electronic phased array.

22. An intravascular observation system comprising at least the catheter according to claim 14,
a fluid delivery device for delivering a transparent fluid to the fluid delivery channel to inject the transparent fluid from the tip of the fluid delivery channel contained in the tubular body of the catheter, and
a control device for controlling driving of the fluid delivery device, wherein
the fluid delivery device is constituted to be controlled by the control device to perform or stop delivery of the fluid to the fluid delivery channel, and
the control device is constituted to receive a signal indicating the heart motion of a living organism to be inserted with the aforementioned catheter as an input signal, and control the fluid delivery device based on the input signal to inject a predetermined amount of the transparent fluid from the tip of the fluid delivery channel of the aforementioned catheter, during the period when the blood flow stops at the tip of the catheter.

23. The intravascular observation system according to claim 22, wherein the imaging channel contained in the tubular body of the aforementioned catheter is constituted to start imaging of the observation target site in synchronization with the timing of injection of the transparent fluid from the fluid delivery channel and perform imaging only for a predetermined time, or
while an imaging channel in a tubular body contained in the catheter is constantly imaging the observation target site, it is constituted such that the control device starts recording of images in synchronization with the timing of the injection of a transparent fluid from the fluid delivery channel and performs recording of images only for a predetermined time.

24. The intravascular observation system according to claim 22, further comprising one or more ultrasonic oscillators, which act as ultrasonic wave transmitting-receiving elements, on the tubular body of the catheter, to enable an ultrasonic diagnosis of the observation object inner wall for which the catheter is inserted.

25. The intravascular observation system according to claim 24, wherein a plurality of ultrasonic oscillators are provided as an electronic phased array.

26. The intravascular observation system according to claim 22, wherein the basic shape of the hood tip is a shape obtained by cutting the cylindrical shape or circular truncated cone shape, which is the basic shape of the hood, with a flat plane forming an angle of 30 degrees-60 degrees to the central axis thereof.

27. The intravascular observation system according to claim 22, wherein the basic shape of the hood is a cylindrical shape having the same outer diameter as that of the tubular body.

28. The intravascular observation system according to claim 22, wherein the basic shape of the hood is a hollow circular truncated cone shape enlarging toward the tip,
the outer diameter of the tip of the tubular body is narrower than the outer diameter of the body from the proximal part to an intermediate part of the tubular body, and the hood extends enlarging forward from the outer periphery of the tip,
the maximum outer diameter of the tip of the hood is the same as the outer diameter of the body from the proximal part to the intermediate part of the tubular body.

29. The intravascular observation system according to claim 28, wherein an endoscope with an imaging channel is inserted in the inside of the tubular body of the catheter, a narrow part of the tip of the tubular body secures at least a fluid delivery channel and holds the body of the aforementioned endoscope.

30. The intravascular observation system according to claim 22, wherein
the bending direction by the bending mechanism and the direction of inclination of the hood opening are related such that the tip of the tubular body part bends toward the direction that brings a line segment connecting A and B closer to parallel to the central axis of the catheter or a direction opposite therefrom, wherein A is a point located at the tip side of the outer periphery of the opening at the tip of the hood and B is a point located at a rear end side thereof.
